(19) United States
(12) Patent Application Publication
(10) Pub. No.: US 2012/0028265 A1
(43) Pub. Date: Feb. 2, 2012

(54) METHODS OF USING RET NANOSENSORS

(76) Inventors: Thijs Kaper, Washington, DC (US); Loren L. Looger, Washington, DC (US); Wolf B. Frommer, Washington, DC (US)

(21) Appl. No.: 12/673,072

(22) PCT Filed: Aug. 8, 2008
(86) PCT No.: PCT/US08/09569

§ 371 Date: Oct. 21, 2011

Related U.S. Application Data
(60) Provisional application No. 60/955,122, filed on Aug. 10, 2007.

(51) Int. Cl. G01N 21/75; G01N 21/76; C12M 1/34; G01N 21/64

(52) U.S. Cl. 435/7.1; 435/288.7

(57) ABSTRACT

The present invention provides methods for detecting and monitoring metabolite concentrations, which comprise detection and measurement of Fluorescence Resonance Energy Transfer upon ligand binding. The methods of the present invention are useful for real time monitoring of changes in metabolite levels in living cell cultures.

### RELATED APPLICATION

This application claims the benefit of U.S. Provisional Application 60/955,122, filed Aug. 10, 2008, incorporated herein in its entirety.

### STATEMENT OF GOVERNMENT SUPPORT

This work was supported by Grant No. NIH R33DK070272. The government may have certain rights to this invention.

### FIELD OF INVENTION

The invention relates generally to methods for measuring and detecting changes in ligand concentration using resonance energy transfer (RET). In particular, the invention provides RET sensors for monitoring metabolic flux of carbohydrates in microorganisms.

### BACKGROUND OF INVENTION

All publications and patent applications herein are incorporated by reference to the same extent as if each individual publication or patent application was specifically and individually indicated to be incorporated by reference.

The publications discussed herein are provided solely for their disclosure prior to the filing date of the present application. Nothing herein is to be construed as an admission that the present invention is not entitled to antedate such publication by virtue of prior invention.

The analysis of the composition of, and changes in the levels of, metabolites and solutes in different compartments of living cells of different tissues or organs is a time-consuming procedure and requires the disruption of the cells. Moreover, most techniques do not measure metabolite and solute changes in real-time and do not take into account temporal and spatial variations of local metabolite concentrations at the cellular or subcellular level. In addition, the methods known in the art have a low resolution and are prone to artifacts.

For instance, little is known about the distribution of arabinose and maltose in different compartments of the cell. No currently available technology addresses these issues in a satisfactory manner. Non-aqueous fractionation is static, invasive, has no cellular resolution and is sensitive to artifacts. While spectroscopic methods such as NMRi (nuclear magnetic resonance imaging) and PET (positron emission tomography) provide dynamic data, they have poor spatial resolution. As one can imagine, no resolution in space is obtained and any information on the distribution of metabolite concentration thereby gets lost. Also, to gain information on the subcellular distribution of metabolites so far requires the analysis of respective compartments after a destructive approach (Bussis et al., Planta (1997) 202, 126-136). With respect to such measurements, there is no tool available that allows for the real-time detection and monitoring of metabolite levels in cells.

The advent of fluorescent proteins has allowed intracellular labeling, especially of peptides, which are easily detectable by optical means. For instance, the green fluorescent protein (GFP) from Aequorea Victoria is a widely used reporter gene in many organisms. Multiple variants with different spectral properties have been developed. Furthermore, combinations of fluorescent proteins exhibiting energy transfer provide for differential fluorescence in response to conformational changes in the protein's immediate environment. However, due to the limitation of the methods described in the prior art, there is an urgent need to provide sensor molecules facilitating the measurement of a broad range of analytes such as organic compounds (e.g. sugars, amino acids, organic acids, vitamins), organic macromolecules and the like. In view of the limitations of the prior art, there is a need for a system and method that allow for the real-time detection and monitoring of changes in metabolite levels in cells.

The development of genetically encoded molecular sensors, which transduce an interaction of the target molecule with a recognition element into a macroscopic observable signal, via allosteric regulation of one or more signaling elements, may provide answers to some of the questions. The recognition element may simply bind the target, bind and enzymatically convert the target, or may serve as a substrate for the target, as in the use of a specific target sequence in the construction of a protease sensor. The most common reporter element is a sterically separated donor-acceptor FRET pair of fluorescent proteins (GFP spectral variants or otherwise) (Fehr, M., Frommer, W. B., and Lalonde, S. 2002, Proceedings of the National Academy of Sciences of the USA 99: 9846-9851) although single fluorescent proteins or enzymes are viable, as well. Some molecular sensors additionally employ a conformational actuator (most commonly a peptide which binds to one conformational state of the recognition element), to magnify the allosteric effect upon and resulting output of the reporter element.

Members of the bacterial periplasmic binding protein superfamily (PBPs) recognize hundreds of substrates with high affinity (atto- to low micro-molar) and specificity. PBPs have been shown by a variety of experimental techniques to undergo a significant conformational change upon ligand binding; fusion of an individual sugar-binding PBP with a pair of GFP variants was used to produce biosensors (Fehr, M., Frommer, W. B., and Lalonde, S. 2002, Proc. Natl. Acad. Sci. 99: 9846-9851). Such sensors have been used to measure sugar uptake and homeostasis in cells, and sub-cellular analyte levels were determined with nuclear-targeted versions. PBPs have been successfully exploited for the construction of fluorescent indicator proteins (FLIPS) for imaging of key metabolites such as glucose (Fehr et al., 2003, J. Biol. Chem. 278: 19127-19133) and glutamate (Okumoto et al., 2005, Proc. Natl. Acad. Sci. U.S.A. 102: 8740-8745). The successful development of biosensors with bacterial PBPs suggests that a similar strategy might be adopted to generate a biosensor to monitor other types of molecules. Until now, biosensors have not been developed for monitoring of intracellular concentrations of molecules in microbial and specifically prokaryotic cells in culture.

### SUMMARY OF INVENTION

The present invention provides methods of detecting and monitoring intracellular metabolites and metabolic flux in cell cultures or integrated over multiple cells. The methods of the present invention yields data with cellular and subcellular resolution and is minimally invasive. The present invention also provides a device for measuring and analyzing changes in resonance energy transfer (RET).

In one embodiment, the present invention provides a method for detecting intracellular levels of a molecule in living cell cultures comprising expressing a nanosensor in the living cells, wherein the nanosensor comprises a donor moiety, an acceptor moiety, and a molecule binding site, and detecting a change in resonance energy transfer between the acceptor moiety and the donor moiety upon binding of the molecule to the molecule binding site, thereby detecting the level of the molecule in the living cell.

In another embodiment, the present invention provides a method of monitoring the intracellular levels of a molecule in living cell cultures comprising expressing a nanosensor in living cells, wherein the nanosensor comprises a donor moiety, an acceptor moiety, and a molecule binding site, detecting a change in resonance energy transfer between the acceptor moiety and the donor moiety, thereby monitoring the intracellular level of the molecule in the living cells.

The present invention also provides a method of monitoring metabolic flux in living cell cultures comprising expressing a nanosensor in living cells, wherein the nanosensor comprises a donor moiety, an acceptor moiety, and a metabolite binding site, and detecting a change in resonance energy between the acceptor moiety and the donor moiety, thereby monitoring the metabolic flux in the cells.

The molecule may be an analyte, a metabolite, a ligand, substrate or any compound that will bind to the molecule binding site (or metabolite binding site). The molecule may be selected from the group consisting of sugars, amino acids, peptides, organic acids, metals or ions, oxides, hydroxides or conjugates thereof, inorganic ions, amines, polyamines and vitamins. The sugars may be selected from the group consisting of arabinose, maltose, glucose, galactose, sucrose, trehalose, fructose, xylose, cellobiose and ribose.

The binding of a molecule to the binding site induces a conformational change in the relative positions of the donor and acceptor moieties which results in a change in resonance energy transfer. The binding site may be in the donor moiety or may be a separate moiety that is coupled to the donor moiety and/or the acceptor moiety. The molecule binding site may be on a protein, such as a periplasmic binding protein (PBP).

The resonance energy transfer (RET) may be fluorescence resonance energy transfer (FRET), phosphorescence resonance energy transfer (PRET), chemiluminescence resonance energy transfer (CRET), or bioluminescence resonance energy transfer (BRET).

The living cell cultures may comprise prokaryotic cells or eukaryotic cells. Examples of prokaryotic cells include but are not limited to bacteria or archaea. Examples of eukaryotic cells include but are not limited to yeast, fungi, protists, and plants

The intracellular levels of the molecule is detected and/or monitored in a real time or on-line time frame. The metabolic flux of living cells in cultures is monitored and/or detected in a real time or on-line time frame.

The method of detecting and/or monitoring the level of an intracellular molecule or metabolic flux of living cell cultures may comprise calculating a ratio of energy emission values detected from the emission of the donor moiety and the emission from the acceptor moiety.

The present invention provides nansosensors for use in the present invention. As an example, the present invention provides an arabinose sensor for monitoring and/or detecting pentose metabolic flux in living cells. The present invention also provides a glucose sensor, for monitoring and/or detecting hexose metabolic flux in living cells. Moreover, the present invention provides a maltose sensor for monitoring and/or detecting dissaccharide flux or glucose flux into maltose.

In one embodiment, the methods of the present invention may be used to detect and/or monitor intracellular levels of molecules or metabolites in living cell cultures, wherein the cells are genetically engineered to produce biofuel. The cells may be prokaryotes or eukaryotes. As an example, the cells may be selected from the group consisting of bacteria, archaea, or eukarya. Examples of eukarya include but are not limited to yeast, fungi, protists, and plants.

The methods of the present invention may be performed in an in vivo system, for example, in a transgenic animal. The method may comprise providing an animal whose transgene encodes a nanosensor. Generally, a transgenic animal is produced by the integration of a given transgene into the genome in a manner that permits the expression of the transgene, or by disrupting the wild-type gene, leading to a knockout of the wild-type gene. Methods for producing transgenic animals are generally described by Wagner and Hoppe (U.S. Pat. No. 4,873,191; which is incorporated herein by reference), Brinster et al. (1985; which is incorporated herein by reference in its entirety) and in “Manipulating the Mouse Embryo; A Laboratory Manual” 2nd edition (eds., Hogan, Beddington, Costantimi and Long, Cold Spring Harbor Laboratory Press, 1994; which is incorporated herein by reference in its entirety).

U.S. Pat. No. 5,639,457 is also incorporated herein by reference for teaching regarding transgenic animals such as transgenic pig and rabbit production. U.S. Pat. Nos. 5,175,384; 5,175,385; 5,530,179, 5,625,125, 5,612,486 and 5,565,186 are also each incorporated herein by reference to similarly for teaching regarding transgenic mouse and rat production.

Additionally, the present invention provides devices for measurement of resonance energy transfer in living cells in a real time or on-line time frame. In one embodiment, the device of the present invention comprises a RET detection unit, a reference unit, and a calibration unit (FIG. 8).

The RET detection unit comprises: a first filter (Filter 1) through which the light from the light source passes to excite a donor moiety in the living cell; a second filter (Filter 2) through which light emitted from the donor moiety passes to reach the detector; a third filter (Filter 3) through which light emitted from the acceptor moiety passes to reach the detector; a first detector (Detector 1) for measuring the emitted light intensity of the donor after excitation of the donor; and a second detector (Detector 2) for measuring the emitted light intensity of the acceptor after excitation of the donor.

The reference unit comprises: a light source for excitation of molecules; a first filter (Filter 3) through which light emitted from the acceptor moiety passes to reach the detector, which is identical to filter 3 in the RET detection unit; and a second filter (Filter 4) through which light emitted from the light source passes to excite an acceptor moiety in the living cell; and a third detector (Detector 3) for measuring the emitted light intensity of the acceptor after excitation of the acceptor.

The calibration unit comprises: an injector with a container containing an analyte stock solution and a valve that can close off the device. The injector is used to calibrate the measurement cell (the device) with external ligands in order to verify the response of the sensor. Though theoretically FRET values are ratiometric and should be independent of sensor levels, optical problems and signal to noise may affect the readout. Thus calibration provides a means of detecting the apo and sat ratios and from that the actual in vivo level in the normal growth conditions can be calculated.

In another embodiment, the device (FIG. 9) comprises: a light source for excitation of molecules; a filter through which the light from the light source passes to excite a donor moiety in the living cells; an optical component that disperses the emitted light from the donor and the acceptor; and a detector for recording the spectrum of the dispersed light.

The light source in the device of the present invention may be selected from the group consisting of a LED, a mercury lamp, a xenon lamp, and a laser.

The detector in the device of the present invention may comprise: at least two photo multiplier detectors; and a processor which processes the signals from the at least two photo multiplier detectors. The photomultiplier detector is a light sensitive diode or a CCD chip. The processor calculates a ratio of light intensity values comprising a ratio of energy emission values detected from the emission of the donor moiety and the emission from the acceptor moiety.

The present invention provides methods of monitoring the concentration of a sugar during fermentation or hydrolysis processes comprising mounting the device of in a fermenter and using the device to monitor the concentration of the sugar.

The present invention also provides a method of monitoring the concentration of a sugar during fermentation or hydrolysis processes comprising passing a fermenter culture through the device and using the device to monitor the concentration of the sugar.

### BRIEF DESCRIPTION OF THE DRAWINGS

FIG. 1 depicts a three-dimensional structure of Escherichia coli L-arabinose binding protein AraF in ribbon representation, with bound arabinose shown in stick representation.

FIG. 2 depicts an in vitro FRET ratio change of the FRET nanosensor FLIParaF.Ec-200n in the presence of D/L-arabinose. The binding constant K_{d} of the FRET nanosensor in the presence of D/L-arabinose is 230±18 nM.

FIG. 3 depicts a graph showing the effect of different concentrations of pentose and hexose substrates on the emission ratio (528 nm over 485 nm) of the FRET nanosensor FLIParaF.Ec-200n.

FIG. 4 depicts a graph showing FLIPara-230n detection of intracellular arabinose in E. coli BL21(DE3) cells at 30° C. Final D/L-arabinose concentrations in the medium in μM. Arrow indicates time point of arabinose addition. Bars indicate standard deviation of at least three replicates.

FIG. 5 depicts a graph showing an arabinose dependent sensor response at t=50 min. Intracellular arabinose concentration corresponding to the affinity constant (K_{d}) of FLIParaF.Ec-200n is reached at an external arabinose concentration of 3.6±1.2 μM, indicating that at that point the intracellular concentration is about 15-fold lower than the extracellular concentration.

FIG. 6 depicts a graph showing FLIPmal-37μ (p3367) detection of intracellular maltose in E. coli BL21(DE3) cells at 30° C. Final maltose concentrations in the medium in μM. Arrow indicates time point of maltose addition. Bars indicate standard deviation of at least three replicates.

FIG. 7 depicts a graph showing a maltose dependent sensor response at t=25 min. Intracellular concentration corresponding to the sensor's affinity constant (K_{d}) is reached at an external maltose concentration of 10.0±2.5 mM, indicating that at that point the intracellular concentration is about 250-fold lower than extracellular concentration.

FIG. 8 depicts a FRET detection device for online monitoring of ECFP-VENUS nanosensor output in cell cultures. The device contains a RET detection unit, a reference detection unit and a calibration unit. Light source emits light of 400-550 nm. Filter 1: 433 nm, Filter 2: 485 nm, Filter 3: 528 nm. Filter 4: 500 nm. BS: Beam splitter. Calibration unit consists of an injector with a reservoir for an analyte stock solution and a valve that can close off the measuring cell.

FIG. 9 depicts an alternative FRET detection device for online monitoring of ECFP-VENUS nanosensor output in cell cultures. The device contains a light source, a 433 nm filter, a light disperser and a detector.

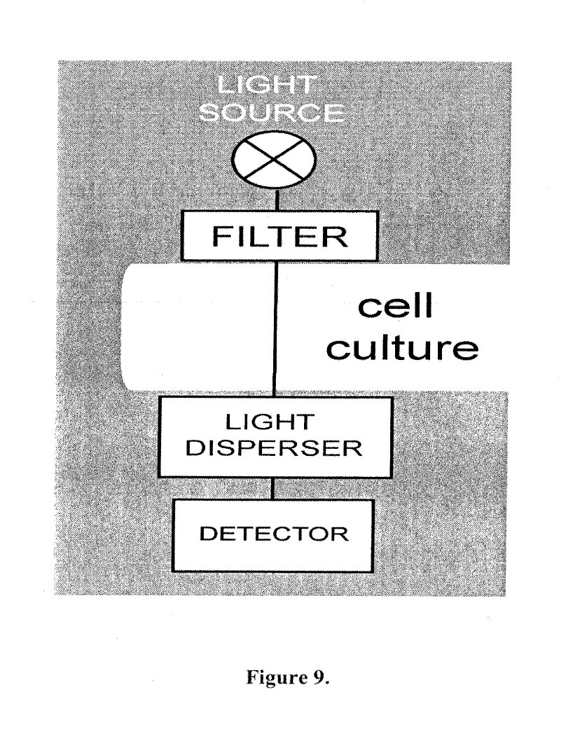

FIGS. 10A-D depict the use of nanosensor for measuring flux changes. (A) Theoretical curve of glucose-induced FRET changes of FLII₁₂Pglu700μ-δ6. S=Δr/ΔRₘₐₓ=[gluc]/([gluc]+K_{d}) with S, nanosenor saturation; Δr, partial ratio change; ΔRₘₐₓ, maximum ratio change; [gluc], glucose concentration; K_{d}, affinity constant of nanosenor (0.66 mM for FLII₁₂Pglu700μ-δ6). Light gray shaded area indicates 10-90% confidence range of FLII₁₂Pglu700μ-δ6 detection range, the dark gray shaded area indicates 20-80% confidence range. (B) Effect of the variation in ratio data (SD) on the determination of glucose concentrations. The solid line shows the variation in ratio data ±2.5% SD, dashed line shows the variation in ratio data ±5% SD. The calculated glucose concentration from the average of ratio data was set as 100%. In vivo glucose concentration was calculated from the equation: [gluc]ᵥᵢᵥₒ=K_{d}×(Δr−1)/(ΔRₘₐₓ−Δr) with [G], glucose concentration; K_{d}, affinity constant of nanosenor; Δr, partial ratio change; ΔRₘₐₓ, maximum ratio change. (C) Examples of in vivo glucose concentration dynamics upon cell perfusion and nanosenor range of FLII₁₂Pglu700μ-δ6. The glucose concentration dynamics within the nanosenor range report differences in 1) accumulation rate, 2) elimination rate, 3) steady-state level, and 4) delay in nanosenor response. Gray lines indicate real in vivo glucose concentration dynamics and black lines indicate the nanosenor saturation of FLII₁₂Pglu700μ-δ6. (D) In vivo glucose concentration-time plot (black line) with ratio ±SD and confidence range (grey lines) of FLII₁₂Pglu700μ-δ6. Beyond the confidence range the SD translates to high variations in apparent glucose concentration.

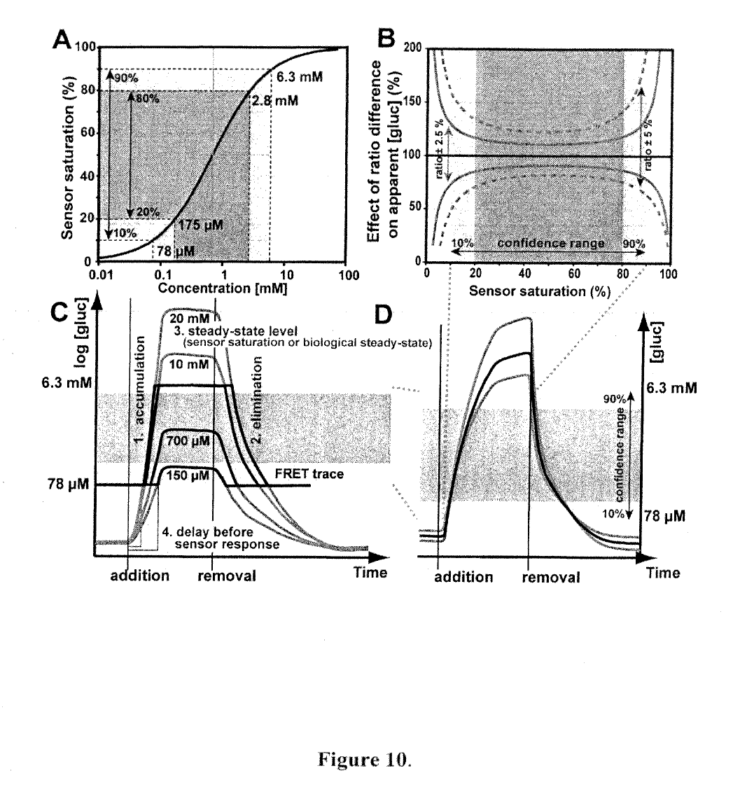

FIGS. 11A and 11B depict the construction and in vitro analysis of improved maltose sensors. (A) Sketches of the original and improved versions of the FLIPmal sensors with shortened linkers. The amino acid sequence in capital letters corresponds to enhanced cyan fluorescent protein/enhanced yellow fluorescent protein or malE; small letters correspond to the synthetic linkers (sequence in bold is the restriction site). (B) In vitro fluorescence resonance energy transfer ratio changes of the FLIPmal sensor variants in the presence of maltose. Error bars represent the standard deviation (n=3).

FIGS. 12A and 12B depict the accumulation of intracellular maltose detected with FLIPmal sensors. (A) Cytosolic maltose accumulation measured in 30-second intervals after injection of buffer or 50 mM maltose to microtiter plate wells containing Escherichia coli BL21-Gold(DE3) cells expressing FLIPmal-40μΔ1-enhanced yellow fluorescent protein. The arrow indicates the time point of maltose addition using the injector of the Tecan Infinite M200 fluorimeter (note, in contrast to FIG. 3A, this procedure allows the determination of rates). (B) Dose-response curve for maltose detected by FLIPmal-40μΔ1-enhanced yellow fluorescent protein in E. coli BL21-Gold(DE3) cells. Intracellular maltose levels are around 700-fold lower compared with the external concentration (K_{0.5}=7.4 mM). Error bars represent the standard deviation (n=3).

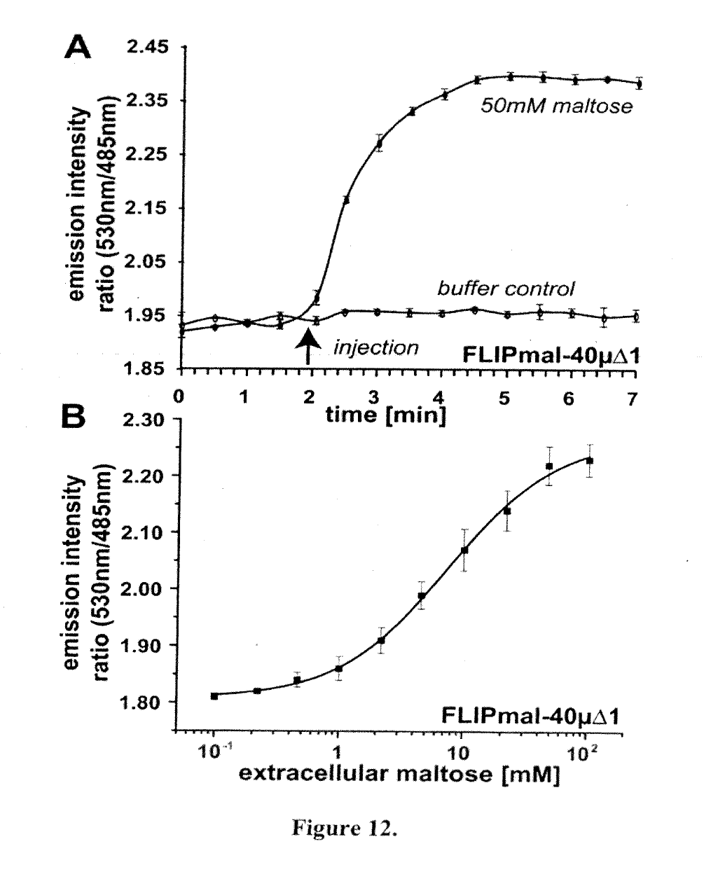

### DETAILED DESCRIPTION OF THE INVENTION

The following description includes information that may be useful in understanding the present invention. It is not an admission that any of the information provided herein is prior art or relevant to the presently claimed inventions, or that any publication specifically or implicitly referenced is prior art.

Other objects, advantages and features of the present invention may become apparent to one skilled in the art upon reviewing the specification and the drawings provided herein. Thus, further objects and advantages of the present invention will be clear from the description that follows.

General Description

The present invention provides non-invasive or minimally invasive methods for detecting or monitoring molecules in a living cell. The present invention is based on resonance energy transfer between molecules as a means for detecting or monitoring molecules in a living cell.

The present invention provides means and methods that allow for real time detection, monitoring and measurement of the concentration of compounds which are not yet accessible by prior art techniques. In one aspect, such means and methods allow for in vivo measurements.

The present inventors have surprisingly found that nanosensors can be used for monitoring intracellular molecule levels in living cells, for example, bacterial cell cultures.

In one aspect, the present invention provides systems for monitoring changes in intracellular ligand concentrations, the system comprising means for monitoring a living cell expressing a ligand binding indicator; and means for analyzing a change in resonance energy transfer, wherein said change in resonance energy transfer indicates a change in level of a ligand in the cell. In one aspect, the ligand binding indicator comprises at least one ligand binding protein moiety; a donor moiety covalently coupled to the ligand binding protein moiety; and an acceptor moiety covalently coupled to the ligand binding protein moiety. In another aspect, the ligand binding indicator includes a ligand binding fluorescent indicator. The change in resonance energy transfer may indicate a change in level of the ligand during a fermentation process. The systems of the present invention are also operable for continuous analysis of the change in resonance energy transfer.

The invention also provides systems for monitoring any type of living cell, for instance, a bacterial cell, and a cell that produces biofuels. In one aspect, monitoring is performed for monitoring the metabolic state of a living cell. In another aspect, monitoring comprises fluorescent spectroscopy. The invention also provides for detection of a change in intracellular concentration of any ligand, including but not limited to sugars, amino acids, peptides, organic acids, metals or ions, oxides, hydroxides or conjugates thereof, inorganic ions, amines, polyamines and vitamins.

The present invention further provides methods of detecting a change in level of a ligand in a living cell, comprising monitoring a living cell expressing a ligand binding indicator, the indicator comprising at least one ligand binding protein moiety, a donor moiety covalently coupled to the ligand binding protein moiety; and an acceptor moiety covalently coupled to the ligand binding protein moiety; and detecting a change in resonance energy transfer between said donor moiety and said acceptor moiety, wherein said change in resonance energy transfer indicates a change in level of a ligand in the cell. The methods of the invention may further comprise analyzing the change in level of the ligand in the cell, including wherein analysis of the change in level of the ligand in the cell indicates a change in metabolism of the ligand in the cell. In one aspect, the step of detecting the change in resonance energy transfer comprises measuring light emitted from the acceptor moiety. In another aspect, the step of detecting the change in resonance energy transfer comprises measuring light emitted from the donor moiety, measuring light emitted from the acceptor moiety, and calculating a ratio of the light emitted from the donor moiety and the light emitted from the acceptor moiety. In yet another aspect, the change in resonance energy transfer indicates a change in intracellular metabolism of the ligand in the cell. In still yet another aspect, the methods of the invention provide continuous monitoring of the cell.

The present invention also provides devices for online measurement of changes in intracellular ligand concentrations in a cell, comprising a monitoring component operable for monitoring a living cell expressing a ligand binding indicator; and an analyzing component for analyzing a change in resonance energy transfer, wherein said change in resonance energy transfer indicates a change in level of a ligand in the cell.

The present invention also provides devices for the detection of intracellular ligand concentrations in a bacterial cell culture, comprising a light source, a filter specific for excitation of an acceptor fluorophore, a filter specific for the excitation of a donor fluorophore, a filter specific for energy emission from the acceptor fluorophore, a filter specific for energy emission from the donor fluorophore, and a detector. In one aspect, the detector comprises at least two photo multiplier detectors; and a processor which processes the signals from the at least two photo multiplier detectors. In another aspect, the processor is operable for calculating a ratio of light intensity values, wherein the ratio comprises a ratio of energy emission values detected from the excitation of the donor fluorophore and the energy emission from the acceptor fluorophore. The light source may, for instance, include an LED, a xenon lamp, or a laser.

The present invention provides methods and systems for monitoring changes in intracellular ligand concentrations in living cells, including a system for monitoring a living cell expressing a ligand binding indicator and for detecting and analyzing a change in resonance energy transfer, wherein a change in resonance energy transfer indicates a change in level of a ligand in the cell. One embodiment, among others, is a system for determining metabolic flux in a living cell, wherein a change in resonance energy transfer is a measure of a change in intracellular concentration, metabolism or flux of the ligand in the cell.

Nanosensors

The present invention provides nanosensors comprising a first detection portion and a second detection portion. The first and second portions are chromophores. The first detection portion may be an energy-absorbing and energy-emitting molecule and the second detection portion may be an energy-absorbing molecule with an excitation peak overlapping with the emission peak of the first molecule, thereby eliciting the second molecule to absorb energy from the first molecule in a non-radiative manner and emit energy in the form of a detectable signal that can also be measured.

The first and second detection portions may be covalently linked. For example, the nanosensors of the present invention may be a fusion protein comprising a first energy-emitting protein and a second energy-absorbing protein. The first and second proteins may be fused by one or more linker peptides. The linker may have any length or amino acid sequence. The linker may be inserted to optimize the fusion protein conformation and improve the energy emission signal.

The first and second portions of the nanosensor may also be linked by chemical conjugation, such that they remain in optimal proximity for resonance energy transfer.

The term “energy-emitting molecule” refers to the first detection portion and comprises molecules capable of radiative energy emission which can (i) take up energy in a suitable form and (ii) transmit at least part of the energy by resonance energy transfer (RET) to the second detection portion, which is thereby elicited to energy emission. The cause for radiative energy emission of the first detection portion may be anything that is conceivable to the person skilled in the art and may involve, for example, a chemical reaction (chemiluminescence or bioluminescence) or absorption of radiation (fluorescence or phosphorescence).

The second detection portions comprise molecules that have an excitation peak that overlap with the energy emitting molecules. Examples of molecules in the second detection portion include chromophores such as fluorescent molecules or fluorescent proteins.

The term “resonance energy transfer” (RET) refers to a non-radiative transfer of excitation energy from a donor molecule (first detection portion) to an acceptor molecule (second detection portion). The conformational change of the nanosensor comprising the donor molecule and the acceptor molecule results in a detectable change of RET between the detection portions. Such a change can for instance be taken from a comparison of the emission spectra of a fusion protein in the absence of a suitable binding compound with the same fusion protein in the presence of such a compound. If, for example, RET is increased, the emission peak of the acceptor is raised and the emission peak of the donor is diminished. Thus, the ratio of the emission intensity of the acceptor to that of the donor is indicative for the degree of RET between the detection portions. The conformational change of the fusion protein upon binding of a compound may result either in a decrease or an increase of the distance between the detection portions. However, not only the distance but also other aspects of the relative position of the detection portions to one another such as the orientation influence RET. Thus, depending on the topology of the detection portions in the nanosensor, RET may increase or decrease upon binding of a compound. In addition, the RET behavior can be determined empirically, e.g., by performing titration experiments.

Depending on the molecule in the first detection portion of the nanosensor, the resonance energy transfer may be fluorescence resonance energy transfer (FRET), bioluminescence resonance energy transfer (BRET), chemiluminescence resonance energy transfer (CRET), and phosphorescence resonance energy transfer (PRET).

The detectable signals generated by the first and second detection portions can be measured using, for example, a fluorimeter or the like, such as a microplate fluorescence spectrophotometer or any other device that selectively measures the intensities of the involved detection portions.

Fluorescent Nanosensors

The present invention provides fluorescent nanosensors for detecting and measuring changes in intracellular levels of specific small molecules using Fluorescence Resonance Energy Transfer (FRET). The fluorescent nanosensors may be used, for instance, for monitoring intracellular metabolite levels in fermentation processes. The terms “biosensor”, “nanosensor”, and “ligand binding indicator” are used interchangeably in the present invention, and each is intended to encompass, for instance, fusion proteins, fluorescent indicator proteins (FLIPs) and any other suitable construct that is operable for continuously monitoring the levels of a ligand in any type of living cell. Moreover, as used herein, the term “ligand binding indicator” includes any construct that comprises at least one ligand binding protein moiety, a donor moiety and an acceptor moiety, wherein a change in resonance energy transfer between the donor moiety and the acceptor moiety indicates ligand binding to the ligand binding protein moiety. In one embodiment, a ligand binding fluorescent indicator includes at least one ligand binding protein moiety; a donor fluorophore moiety covalently coupled to the ligand binding protein moiety; and an acceptor fluorophore moiety covalently coupled to the ligand binding protein moiety, wherein detection of a change in FRET between the donor moiety and the acceptor moiety indicates ligand binding to the ligand binding protein moiety.

According to one embodiment, a change in resonance energy transfer is detected as a change in energy emission, for instance, a change in energy emission ratios, as described below with regard to the example of detection and analysis of fluorescent emission ratios by a fluorescence emission ratio analyzer. The term “energy emission” refers to an optical signal, which may be in the visible spectrum of light, that can be detected by suitable devices known in the art. A change of energy emission may include a significant change, i.e., an increase or decrease, of light intensity at a given wavelength or a significant change in the ratio of the light intensities at two or more different wavelengths compared with each other (also referred to as “ratiometric measurements”). According to one embodiment, a nanosensor of the invention provides a change of light intensity or of the ratio between light intensities at two or more different wavelengths of at least 0.01%, preferentially at least 0.1%, more preferably at least 1%, still more preferably at least 2%, even more preferably at least 5%, and most preferably at least 10% of the state where the ligand is not bound to the ligand binding portion compared to the bound state. Analysis of energy emission values or energy emission ratios may be carried out by comparing the energy emission values with a standard curve showing the relationship of ligand or analyte concentration and energy emission.

The term “continuous monitoring” as used herein is understood to refer to real-time or, alternatively, substantially real-time monitoring. The term “ligand” as used herein is understood to refer to any molecule that enters the cell, is present within the cell, or that leaves the cell, including but not limited to any substrate or metabolite. For instance, the ligand glucose may be continuously monitored for any change in intracellular glucose concentrations, for instance, as a result of glycolysis or any another biochemical process within the cell that utilizes glucose. Such continuous monitoring provides surprisingly valuable information about real-time changes in uptake and efflux of the ligand from a living cell, as well as information about real-time changes in the biosynthesis, metabolism and degradation of the ligand, e.g., metabolic flux, and other utilization, in a living cell. In one embodiment, a fluorescent ligand binding indicator may comprises at least one ligand binding protein moiety; a donor fluorophore moiety covalently coupled to the ligand binding protein moiety; and an acceptor fluorophore moiety covalently coupled to the ligand binding protein moiety; wherein detection of a change in resonance energy transfer between the donor moiety and the acceptor moiety indicates ligand binding to the ligand binding protein moiety.

As used herein, “covalently coupled” means that the donor and acceptor fluorescent moieties may be conjugated to the ligand binding protein moiety via a chemical linkage, for instance to a selected amino acid in said ligand binding protein moiety. Covalently coupled also means that the donor and acceptor moieties may be genetically fused to the ligand binding protein moiety such that the ligand binding protein moiety is expressed as a fusion protein comprising the donor and acceptor moieties. As described herein, the donor and acceptor moieties may be fused to the termini of the ligand binding protein moiety or to an internal position within the ligand binding protein moiety so long as FRET between the donor moiety and the acceptor moiety is altered when the donor moiety is excited and the ligand binds to the ligand binding protein moiety.

The term “fluorescence resonance energy transfer” (FRET) refers to a non-radiative transfer of excitation energy from a donor (first detection portion) to an acceptor molecule (second detection portion). FRET requires donor and acceptor fluorophores with overlapping emission and excitation spectra, respectively. After excitation of the donor, energy is transmitted to the acceptor in a non-radiative manner and emitted by the acceptor. The efficiency of this process depends on the distance between and relative orientation of the dipoles of the fluorophores. Ligand-binding induced conformational changes in the sensors may result in altered FRET efficiencies, which correlate with the levels of the respective metabolites.

Upon binding a ligand which induces a conformational change of the ligand binding protein moiety of a nanosensor, a detectable change of FRET between the detection portions is detected. Such a change can for instance be determined from a comparison of the emission spectra of a nanosensor in the absence of a suitable binding compound with the same nanosensor in the presence of such a compound. If, for example, FRET is increased, the emission peak of the acceptor is raised and the emission peak of the donor is diminished. In one embodiment, the change of the energy emitted by the detection portions is an increase or decrease of FRET. In FRET, both donor and acceptor, i.e. both detection portions, are fluorescent protein or molecule portions and, for measuring FRET, the nanosensor is supplied with energy, i.e. radiation, appropriate for exciting energy emission by the first detection portion.

In one embodiment, the invention provides FRET nanosensors which are proteins that consist of a ligand-binding recognition element and fluorophore reporter elements. The invention also provides isolated nucleic acids encoding the ligand-binding recognition element and fluorophore reporter elements. The invention also provides FRET nanosensors which are fusion proteins comprising two detection portions and a periplasmic binding protein (PBP) portion between these two detection portions which undergoes a conformational change upon binding of a compound.

In another embodiment, the nanosensors of the present invention include a fusion protein comprising two detection portions, wherein the first detection portion is an energy-emitting protein portion and the second detection portion is a fluorescent protein portion; or the two detection portions are portions of a split fluorescent protein; and a PBP portion between said two detection portions, which undergoes a conformational change upon binding of a compound, wherein the conformational change results in a change of the energy emitted by the two detection portions.

The ligand binding indicators of the present invention are also useful for detecting analytes in any given liquid. For instance, the fluorescent indicators are useful for continuous monitoring of intracellular levels of specific molecules in living cells during cultivation and/or continuous monitoring of specific molecule levels in solution. The term “analyte” as used in connection with the present invention refers to ligands, compounds or molecules that can be bound by a PBP and generate a conformational change in the PBP. It has been found that such a conformational change of a PBP can be utilized in that it can change the relative position, i.e. the distance, orientation and/or the spatial relationship, of two detection portions which are fused to the PBP in a way that an energy emission generated by the detection portions is detectably altered. The term “and/or” wherever used herein includes the meaning of “and”, “or” and “all or any other combination of the elements connected by said term.”

For intracellular analyte measurements, the nanosensors or nucleotide sequences encoding said nanosensors of the invention may be transferred into a cell by transfection, transformation, direct microinjection or by microinjection of RNA encoding the nanosensors and capable of expressing it in the cell. Apart from the way of introducing the nanosensors into the cell, this embodiment corresponds to the method for detecting an analyte in a cell described above, wherein the cells are genetically engineered with an nucleic acid molecule, expression construct or vector encoding and capable of expressing the nanosensor in the cell.

According to the present invention, in principle any kind of cell that is amenable to optical detection and That can be transformed or transfected so as to express a heterologous protein may be used in the present invention. Thus, the cells may be bacteria, yeasts, protozoa, archaea, or cultured cells, for example of vertebrate, such as mammalian, including human origin, or plant cells. For certain applications, it may be useful to take pathogenetically affected cells such as tumor cells or cells infected by an infectious agent, e.g. a virus, wherein measurements are conducted in comparison with corresponding healthy cells. Likewise, the cells may be part of a tissue, organ or organism. The cells may also be immobilized which facilitates their observation.

The term “periplasmic binding protein” or “PBP” refers to proteins that are characterized by a three-dimensional lobe-hinge-lobe structure and which upon binding of a ligand undergo a conformational change sufficient and suitable for ligand detection as described herein. The lobes are globular to ellipsoid. The hinge region may contain two or more amino acid strands. This structure is furthermore characterized by the presence of a cleft between the lobes, which is the site of substrate binding, i.e. binding of the compound to be analyzed. Upon binding of a compound, the PBP undergoes a conformational change, whereby the two lobes change relative positions, a movement which is also often referred to as that of a Venus flytrap or Pacman or as a hinge-twist motion. Advantageously, the PBP for use in a fusion protein of the invention has no enzymatic activity. Table 1 shows examples of PBPs from which the three-dimensional structure is already known. In addition, proteins of Table 1, which have not yet been characterized structurally, can be modelled on the basis of the existing structures according to methods known in the art. The PBP useful for the biosensor of the invention has the same three-dimensional structure as these examples or is similar thereto. Thus, they show the above mentioned lobe-hinge-lobe structure and the conformational change upon binding of a compound. The person of average skill in analyzing three-dimensional protein structures knows how to identify proteins having such a lobe-hinge-lobe structure. It is furthermore possible for the person skilled in the art to identify among the proteins that match these structural criteria those who show a conformational change as mentioned, above. For this purpose, he/she can compare three-dimensional structure data from a protein having bound its substrate with data from one not bound. Methods for determining the three-dimensional structure of proteins are well known to the person skilled in the art and are described in the literature such as in Mancini, Structure (1997) 5, 741-750 (cryo-electron microscopy), Qian, Biochemistry (1998) 37, 9316-9322 (NMR), Shilton, J. Mol. Biol. (1996) 264, 350-363 (modeling on the basis of related structures) and Spurlino, J. Biol. Chem. (1991) 266, 5202-5219 (X-ray crystallography).

PBPs may be utilized for the construction of the biosensors of the present invention, including the construction of fluorescent indicator proteins (FLIPS) for imaging of key metabolites. The PBP utilized for the construction of the biosensors according to the present invention may belong to one of the protein superfamilies “periplasmic binding protein like I” (PBP-like I), “periplasmic binding protein like II” (PBP-like II) or “helical backbone” metal receptor superfamily according to the nomenclature of the structural classification of proteins (SCOP; Murzin, J. Mol. Biol. 247 (1995), 536-540), to the families as defined by Saier et al., 1993 Microbiol Rev. 57, 320-346 or to the binding proteins classified as being “ATP-dependent” in the compilation of transport proteins shown under www.biology.ucsd.edu/.about.ipaulsen/transport.

The PBPs that may be utilized for the construction of the biosensors of the present invention may be naturally occurring PBPs. Such PBPs may originate, for instance, from gram-negative bacteria or from gram-positive bacteria. Examples of such PBPs are shown in Table 1.

TABLE 1
Bacterial Periplasmic Binding Proteins

| Gene | | | | |
|---|---|---|---|---|
| name | Substrate | Species | 3D | Reference |
| AccA | agrocinopine | Agrobacterium sp. | —/— | J. Bacteriol. (1997) 179, 7559-7572 |
| AgpE | alpha-glucosides | Rhizobium meliloti | —/— | J. Bacteriol. (1999) 181, 4176-4184 |
| | (sucrose, maltose, | | | |
| | trehalose) | | | |
| AlgQ2 | alginate | Sphingomonassp. | —/c | J. Biol. Chem. (2003) 278, 6552-6559 |
| AlsB | allose | E. coli | —/c | J. Bacteriol. (1997) 179, 7631-7637 |
| | | | | J. Mol. Biol. (1999) 286, 1519-1531 |
| AraF | arabinose | E. coli | —/c | J. Mol. Biol. (1987) 197, 37-46 |
| | | | | J. Biol. Chem. (1981) 256, |
| | | | | 13213-13217 |
| AraS | arabinose/fructose/ | Sulfolobus | —/— | Mol. Microbiol. (2001) 39, 1494-1503 |
| | xylose | solfataricus | | |
| ArgT | lysine/arginine/ | Salmonella | o/c | Proc. Natl. Acad. Sci. USA (1981) |
| | ornithine | typhimurium | | 78, 6038-6042 |
| | | | | J. Biol. Chem. (1993) 268, |
| | | | | 11348-11355 |
| ArtI | arginine | E. coli | | Mol. Microbiol. (1995) 17, 675-686 |
| ArtJ | arginine | E. coli | | Mol. Microbiol. (1995) 17, 675-686 |
| b1310 | (putative, multiple | E. coli | —/— | NCBI accession A64880 |
| | sugar) | | | |
| b1487 | (putative, | E. coli | —/— | NCBI accession B64902 |
| | oligopeptide | | | |
| | binding) | | | |
| b1516 | (sugar binding | E. coli | —/— | NCBI accession G64905 |
| | protein homolog) | | | |
| BtuF | vitamin B12 | E. coli | —/— | J. Bacteriol. (1986) 167, 928-934 |
| CAC1474 | proline/glycine/betaine | Clostridium | —/— | NCBI accession AAK79442 |
| | | acetobutylicum | | |
| cbt | dicarboxylate | E. coli | —/— | J. Supramol. Struct. (1977) 7, 463-80 |
| | (succinate, | | | J. Biol. Chem. (1978) 253, 7826-7831 |
| | malate, fumarat) | | | J. Biol. Chem. (1975) 250, 1600-1602 |
| CbtA | cellobiose | Sulfoblobus | —/— | Mol. Microbiol. (2001) 39, 1494-1503 |
| | | solfataricus | | |
| ChvE | sugar | Agrobacterium | —/— | J. Bacteriol. (1990) 172, 1814-1822 |
| | | tumefaciens | | |
| CysP | thiosulfate | E. coli | —/— | J. Bacteriol. (1990) 172, 3358-3366 |
| DctP | C4-dicarboxylate | Rhodobacter | —/— | Mol. Microbiol. (1991) 5, 3055-3062 |
| | | capsulatus | | |
| DppA | dipeptides | E. coli | o/c | Biochemistry (1995) 34, |
| | | | | 16585-16595 |
| FbpA | iron | Neisseria | —/c | J. Bacteriol. (1996) 178, 2145-2149 |
| | | gonorrhoeae | | |
| FecB | Fe(III)-dicitrate | E. coli | | J. Bacteriol. (1989) 171, 2626-2633 |
| FepB | enterobactin-Fe | E. coli | —/— | J. Bacteriol. (1989) 171, 5443-5451 |
| | | | | Microbiology (1995) 141, 1647-1654 |
| FhuD | ferrichydroxamate | E. coli | —/c | Mol. Gen. Genet. (1987) 209, 49-55 |
| | | | | Nat. Struct. Biol. (2000) 7, 287-291 |
| | | | | Mol. Gen. Genet. (1987) 209, 49-55 |
| FliY | cystine | E. coli | —/— | J. Bacteriol. (1996) 178, 24-34 |
| | | | | NCBI accession P39174 |
| GlcS | glucose/galactose/ | Sulfolobus | —/— | Mol. Microbiol. (2001) 39, 1494-1503 |
| | mannose | solfataricus | | |
| GlnH | glutamine | E. coli | o/— | Mol. Gen. Genet. (1986) 205, 260-9 |
| (protein: | | | | J. Mol. Biol. (1996) 262, 225-242 |
| GLNBP) | | | | J. Mol. Biol. (1998) 278, 219-229 |
| GntX | gluconate | E. coli | —/— | J. Basic. Microbiol. (1998) 38, |
| | | | | 395-404 |
| HemT | haemin | Yersinia | —/— | Mol. Microbiol. (1994) 13, 719-732 |
| | | enterocolitica | | |
| HisJ | histidine | E. coli | —/c | Biochemistry (1994) 33, 4769-4779 |
| (protein: | | | | |
| HBP) | | | | |
| HitA | iron | Haemophilus | o/c | Nat. Struct. Biol. (1997) 4, 919-924 |
| | | influenzae | | Infect. Immun. (1994) 62, 4515-25 |
| | | | | J. Biol. Chem. (195) 270, |
| | | | | 25142-25149 |
| LivJ | leucine/valine/ | E. coli | —/c | J. Biol. Chem. (1985) 260, 8257-8261 |
| | isoleucine | | | J. Mol. Biol. (1989) 206, 171-191 |
| LivK | leucine | E. coli | —/c | J. Biol. Chem. (1985) 260, 8257-8261 |
| (protein: | | | | J. Mol. Biol. (1989) 206, 193-207 |
| L-BP) | | | | |
| MalE | maltodextrine/ | E. coli | o/c | Structure (1997) 5, 997-1015 |
| (protein: | maltose | | | J. Bio.l Chem. (1984) 259, 10606-13 |
| MBP) | | | | |
| MglB | glucose/galactose | E. coli | —/c | J. Mol. Biol. (1979) 133, 181-184 |
| (protein: | | | | Mol. Gen. Genet. (1991) 229, |
| GGBP) | | | | 453-459 |
| ModA | molybdate | E. coli | —/c | Nat. Struct. Biol. (1997) 4, 703-707 |
| | | | | Microbiol. Res. (1995) 150, 347-361 |
| MppA | L-alanyl-gamma-D- | E. coli | | J. Bacteriol. (1998) 180, 1215-1223 |
| | glutamyl-meso-di | | | |
| | aminopimelate | | | |
| NasF | nitrate/nitrite | Klebsiella oxytoca | —/— | J. Bacteriol. (1998) 180, 1311-1322 |
| NikA | nickel | E. coli | —/— | Mol. Microbiol. (1993) 9, 1181-1191 |
| opBC | choline | Bacillus subtilis | —/— | Mol. Microbiol. (1999) 32, 203-216 |
| OppA | oligopeptide | Salmonella | o/c | Biochemistry (1997) 36, 9747-9758 |
| | | typhimurium | | Eur. J. Biochem. (1986) 158, 561-567 |
| PhnD | alkylphosphonate | E. coli | —/— | J. Biol. Chem. (1990) 265, 4461-4471 |
| PhoS | phosphate | E. coli | —/c | J. Bacteriol. (1984) 157, 772-778 |
| (Psts) | | | | Nat. Struct. Biol. (1997) 4, 519-522 |
| PotD | putrescine/ | E. coli | —/c | J. Biol. Chem. (1996) 271, 9519-9525 |
| | spermidine | | | |
| PotF | polyamines | E. coli | —/c | J. Biol. Chem. (1998) 273, |
| | | | | 17604-17609 |
| ProX | betaine | E. coli | | J. Biol. Chem. (1987) 262, |
| | | | | 11841-11846 |
| rbsB | ribose | E. coli | o/c | J. Biol. Chem. (1983) 258, 12952-6 |
| | | | | J. Mol. Biol. (1998) 279, 651-664 |
| | | | | J. Mol. Biol. (1992) 225, 155-175 |
| SapA | peptides | Salmonella | —/— | EMBO J. (1993) 12, 4053-4062 |
| | | typhimurium | | |
| Sbp | sulfate | Salmonella | —/c | J. Biol. Chem. (1980) 255, 4614-4618 |
| | | typhimurium | | Nature (1985) 314, 257-260 |
| TauA | taurin | E. coli | —/— | J. Bacteriol. (1996) 178, 5438-5446 |
| TbpA | thiamin | E. coli | —/— | J. Biol. Chem. (1998) 273, 8946-8950 |
| TctC | tricarboxylate | Salmonella | —/— | |
| | | typhimurium | | |
| ThuE | trehalose/maltose/ | Sinorhizobium | —/— | J. Bacteriol. (2002) 184, 2978-2986 |
| | sucrose | meliloti | | |
| TreS | trehalose | Sulfolobus | —/— | Mol. Microbiol. (2001) 39, 1494-1503 |
| | | solfataricus | | |
| tTroA | zinc | Treponema | —/c | Gene (1997) 197, 47-64 |
| | | pallidum | | Nat. Struct. Biol. (1999) 6, 628-633 |
| UgpB | sn-glycerol-3-phosphate | E. coli | —/— | Mol. Microbiol. (1988) 2, 767-775 |
| XylF | xylose | E. coli | —/— | Receptors Channels (1995) 3, |
| | | | | 117-128 |
| YaeC | unknown | E. coli | —/— | J Bacteriol (1992) 174, 8016-22 |
| | | | | NCBI accession P28635 |
| YbeJ | glutamate/aspartate | E. coli | —/— | NCBI accession E64800 |
| (GltI) | (putative, | | | |
| | superfamily: | | | |
| | lysine-arginine-ornithine- | | | |
| | binding | | | |
| | protein) | | | |
| YdcS | (putative, | E. coli | —/— | NCBI accession P76108 |
| (b1440) | spermidine) | | | |
| YehZ | unknown | E. coli | —/— | NCBI accession AE000302 |
| YejA | (putative, | E. coli | —/— | NCBI accession AAA16375 |
| | homology to | | | |
| | periplasmic | | | |
| | oligopeptide-binding | | | |
| | protein— | | | |
| | Helicobacter | | | |
| | pylori) | | | |
| YgiS | oligopeptides | E. coli | —/— | NCBI accession Q46863 |
| (b3020) | | | | |
| YhbN | unknown | E. coli | —/— | NCBI accession P38685 |
| YhdW | (putative, amino | E. coli | —/— | NCBI accession AAC76300 |
| | acids) | | | |
| YliB | (putative, | E. coli | —/— | NCBI accession P75797 |
| (b0830) | peptides) | | | |
| YphF | (putative sugars) | E. coli | —/— | NCBI accession P77269 |
| Ytrf | acetoin | B. subtilis | —/— | J. Bacteriol. (2000) 182, 5454-5461 |
| ZnuA | zinc | Synechocystis | —/— | J. Mol. Biol. (2003) 333, 1061-1069 |

Table 1: Periplasmic binding proteins suitable for making nanosensors. Where appropriate, the species identified is the one for which the 3D-structure is known. At present, the 3D structure of PBPs are mostly known from E. coli and S. typhimurium. These structures are shown at http://scop.mrc-lmb.cam.ac.uk/scop/index.htm or http://ncbi.nlm.nih.gov. The column headed “3D” indicates whether three-dimensional, open (“o”) or closed (“c”) structures of the respective PBP are available in the literature.

Bacterial PBPs have the ability to bind a variety of different molecules and nutrients, including sugars, amino acids, vitamins, minerals, ions, metals and peptides, as shown in Table 1. Thus, PBP-based ligand binding sensors may be designed to permit detection and quantitation of any of these molecules according to the methods of the present invention. Naturally occurring species variants of the PBPs listed in Table 1 may also be used, in addition to artificially engineered variants comprising site-specific mutations, deletions or insertions that maintain measurable ligand binding function.

Likewise, homologues of the PBPs mentioned in Table 1 may be used for carrying out the present invention such as orthologues originating from related species of gram-negative bacteria. Furthermore, encompassed by the term “PBP” are functional analogues of the PBPs of gram-negative bacteria. Such functional analogues likewise show a lobe-hinge-lobe structure, they can bind compounds and show a conformational change upon compound binding as described above. For instance, such functional analogues include homologues of PBPs from gram-negative bacteria which are found in gram-positive bacteria (Quiocho, Mol. Microbiol. 20 (1996), 17-25; Gilson, EMBO J. 7 (1988), 3971-3974; Turner, J. Bacteriol (1999) 181: 2192-2198). Further examples of functional PBP analogs comprise proteins belonging to the above-mentioned superfamilies, which are not present in the periplasm of gram-negative bacteria such as the amide receptor/negative regulator of the amidase operon (AmiC) from Pseudomonas aeruginosa, the lac-repressor (lacR) core C-terminal domain from E. coli as examples of the PBP-like I superfamily and thiaminase I from Bacillus subtilis or the putrescine receptor (potF) from E. coli as examples of the PBP-like II superfamily.

Moreover, it is contemplated that the term “PBP” includes derivatives of any one of the above-mentioned periplasmic binding proteins as long as such derivatives have the above-described lobe-hinge-lobe structure, binding capacity and suitable conformational change upon binding of a compound. It is also to be understood that the skilled person is capable of modifying and optimizing naturally occurring PBPs by suitable techniques known in the art such as in vitro or in vivo mutagenesis, PCR shuffling mutagenesis, chemical modification and the like.

The term “compound” (also designated “analyte” or “ligand” or “molecule” or “substrate” throughout the present description) refers to any compound that can be bound by the ligand binding protein moiety, such as the PBP portion described above. Depending on the compound, ligand or analyte that is to be detected, a suitable ligand binding protein moiety or PBP can be selected to construct the biosensor according to the present invention. Examples of compounds detectable by the biosensors of the invention are given in Table 1.

The term “binding of a compound” refers, in one aspect, to non-covalent binding of a compound to a ligand binding protein moiety, such as the PBP portion which triggers a conformational change of the PBP portion as described above. Such binding may involve non-covalent interactions such as salt bridges, hydrogen bonds, van der Waal forces, stacking forces, complex formation or combinations thereof between the compound and the ligand binding protein moiety, such as the PBP portion described above. It may also include interactions with water molecules in the binding pocket.

The present invention also relates to nucleic acid molecules comprising a nucleotide sequence encoding such biosensors as well as to expression cassettes, vectors, and host cells comprising said nucleic acid molecules. One embodiment is an isolated nucleic acid which encodes an arabinose FRET nanosensor, the nanosensor comprising: an arabinose binding protein moiety, a donor fluorescent protein moiety covalently coupled to the arabinose binding protein moiety, and an acceptor fluorescent protein moiety covalently coupled to the arabinose binding protein moiety, wherein FRET between the donor moiety and the acceptor moiety is changed when the donor moiety is excited and arabinose binds to the arabinose binding protein moiety. FRET nanosensors according to the invention can thus be used for the specific detection of the pentose arabinose and such sensors can also successfully be used to monitor flux of arabinose and maltose in bacteria, for instance, E. coli using fluorescence spectroscopy.

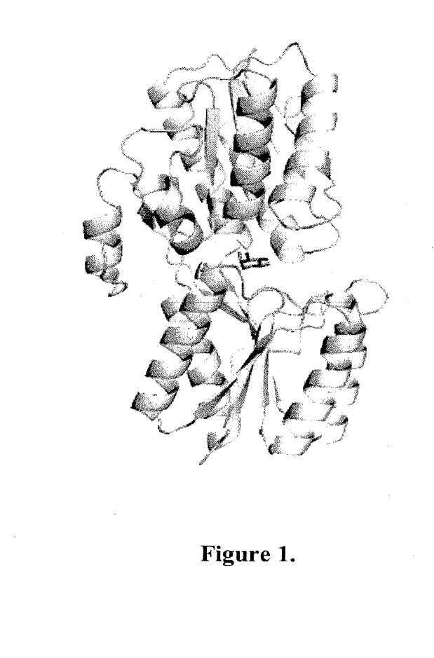

One example of a ligand binding protein moiety, among others, is a high-affinity arabinose binding protein AraF from E. coli. AraF is a polypeptide consisting of 306 amino acid residues that fold in two well-defined lobes connected by a flexible hinge region (FIG. 1) (Quiocho, F. A. and Vyas, N. K., 1984, Nature 310(5976), 381-6). The ligand-binding site is located at the interface between the two lobes (Quiocho, F. A. and Vyas, N. K., 1984, Nature 310(5976), 381-6). Any portion of the AraF sequence which encodes an arabinose binding region may be used in the nucleic acids of the present invention. Arabinose binding portions of AraF or any of its homologues from other organisms may be cloned into the vectors described herein and screened for activity according to the disclosed assays.

Naturally occurring species variants of an arabinose binding protein, such as AraF, may also be used, in addition to artificially engineered variants comprising site-specific mutations, deletions or insertions that maintain measurable arabinose binding function. Variant nucleic acid sequences may, for instance, have at least 30, 40, 50, 60, 70, 75, 80, 85, 90, 95, or 99% similarity or identity to the gene sequence for AraF. Suitable variant nucleic-acid sequences may also hybridize to the gene for AraF under highly stringent hybridization conditions. High stringency conditions are known in the art; see for example Maniatis et al., Molecular Cloning: A Laboratory Manual, 2d Edition, 1989, and Short Protocols in Molecular Biology, ed. Ausubel, et al., both of which are hereby incorporated by reference. Stringent conditions are sequence-dependent and will be different in different circumstances.

Artificial variants of the present invention may be designed to exhibit decreased affinity for the ligand, in order to expand the range of ligand concentration that can be measured by the disclosed nanosensors. Additional artificial variants showing decreased or increased binding affinity for ligands may be constructed by random or site-directed mutagenesis and other known mutagenesis techniques, and cloned into vectors and screened for activity. The binding specificity of the nanosensors may also be altered by mutagenesis so as to alter the ligand recognized by the nanosensor. See, for instance, Looger et al., 2003, Nature, 423 (6936): 185-190.

The nanosensors of the invention may also be designed, for instance, with an arabinose or maltose binding moiety and one or more additional ligand binding moieties that are covalently coupled or fused together and to the donor and acceptor fluorescent moieties in order to generate an allosteric enzyme whose activity is controlled by more than one ligand. Allosteric enzymes containing dual specificity for more than one ligand have been described in the art, and may be used to construct the FRET nanosensors described herein (Guntas and Ostermeier, 2004, J. Mol. Biol. 336(1): 263-73).

The nanosensors of the invention may incorporate any suitable donor and acceptor fluorescent protein moieties that are capable in combination of serving as donor and acceptor moieties in FRET. Examples of donor and acceptor moieties include those selected from the group consisting of GFP (green fluorescent protein), CFP (cyan fluorescent protein), BFP (blue fluorescent protein), YFP (yellow fluorescent protein), and enhanced variants thereof, with one embodiment provided by the donor/acceptor pair eCFP/YFP Venus, or eCFP/Venus, in which Venus is a variant of YFP with improved pH tolerance and maturation time (Nagai, T., Ibata, K., Park, E. S., Kubota, M., Mikoshiba, K., and Miyawaki, A. (2002) Criteria to consider when selecting donor and acceptor fluorescent moieties is known in the art, for instance as disclosed in U.S. Pat. No. 6,197,928, which is herein incorporated by reference in its entirety.

When the fluorophores of the nanosensor contain stretches of similar or related sequence(s), gene silencing may adversely affect expression of the nanosensor in certain cells and particularly whole organisms. In such instances, it is possible to modify the fluorophore coding sequences at one or more degenerate or wobble positions of the codons of each fluorophore, such that the nucleic acid sequences of the fluorophores are modified but not the encoded amino acid sequences. One or more conservative substitutions that do not adversely affect the function of the fluorophores may also be incorporated.

The invention further provides vectors, including expression vectors, containing isolated nucleic acid molecules encoding the nanosensors, and host cells comprising nucleic acids, as well as the nanosensor proteins encoded by the nucleic acids. Such nucleic acids, vectors, host cells and proteins may be used, for instance, in methods of detecting changes in analyte levels, for monitoring changes or flux in intracellular molecule levels in fermentation processes, and for monitoring of intracellular levels of specific molecules in living cells during cultivation or for continuous monitoring of specific molecule levels in solutions using the nanosensors described herein.

Exemplary vectors include vectors derived from a virus, such as a bacteriophage, a baculovirus or a retrovirus, and vectors derived from bacteria or a combination of bacterial sequences and sequences from other organisms, such as a cosmid or a plasmid. Such vectors include expression vectors containing expression control sequences operatively linked to the nucleic acid sequence coding for the biosensor. Vectors may be adapted for function in a prokaryotic cell, such as E. coli or other bacteria, or a eukaryotic cell, including animal cells or plant cells. For instance, the vectors of the invention will generally contain elements such as an origin of replication compatible with the intended host cells, one or more selectable markers compatible with the intended host cells and one or more multiple cloning sites. The choice of particular elements to include in a vector will depend on factors such as the intended host cells, the insert size, whether regulated expression of the inserted sequence is desired, i.e., for instance through the use of an inducible or regulatable promoter, the desired copy number of the vector, the desired selection system, and the like. The factors involved in ensuring compatibility between a host cell and a vector for different applications are well known in the art. In one embodiment, a DNA sequence that encodes the nanosensor is integrated in a plasmid, which in addition contains a selective marker and transcription and translation start sequences, as well as optional subcellular targeting sequences.

The invention also includes host cells transfected with a vector or an expression vector of the invention, including prokaryotic cells, such as E. coli or other bacteria, or eukaryotic cells, such as yeast cells, animal cells or plant cells.

In one embodiment, vectors for use in the present invention will permit cloning of the arabinose or maltose binding domain or protein moiety between nucleic acids encoding donor and acceptor fluorescent molecules, resulting in expression of a chimeric or fusion protein comprising, for instance, the arabinose binding domain covalently coupled to donor and acceptor fluorescent molecules. Exemplary vectors include the bacterial pRSET-FLIP derivatives disclosed in Fehr et al. (2002) (Visualization of maltose uptake in living yeast cells by fluorescent nanosensors, Proc. Natl. Acad. Sci. USA 99: 9846-9851), which is herein incorporated by reference in its entirety. Methods of cloning nucleic acids into vectors in the correct frame so as to express a fusion protein are well known in the art.

The nanosensors of the present invention may be expressed in any location in the cell, including the cytoplasm, cell surface or subcellular organelles such as the nucleus, vesicles, ER, vacuole, etc. Methods and vector components for targeting the expression of proteins to different cellular compartments are well known in the art, with the choice dependent on the particular cell or organism in which the biosensor is expressed. See, for instance, Okumoto, S., Looger, L. L., Micheva, K. D., Reimer, R. J., Smith, S. J., and Frommer, W. B. (2005) Proc. Natl. Acad Sci USA 102(24): 8740-8745; Fehr, M., Lalonde, S., Ehrhardt, D. W., and Frommer, W. B. (2004) J. Fluoresc. 14(5), 603-609, which are herein incorporated by reference in their entireties.

According to another embodiment, the nanosensors of the present invention may be constructed such that the donor and acceptor fluorescent moiety coding sequences are fused to separate termini of the ligand binding domain in a manner such that changes in FRET between donor and acceptor may be detected upon ligand binding. Fluorescent domains can optionally be separated from the ligand binding domain by one or more flexible linker sequences. In one embodiment, such linker moieties are preferably between about 1 and 50 amino acid residues in length, and more preferably between about 1 and 30 amino acid residues. Linker moieties and their applications are well known in the art and described, for example, in U.S. Pat. Nos. 5,998,204 and 5,981,200, and Newton et al., Biochemistry 35:545-553 (1996). Alternatively, shortened versions of linkers or any of the fluorophores described herein may be used.

It will also be possible depending on the nature and size of the binding domain, for instance, an arabinose binding domain, to insert one or both of the fluorescent molecule coding sequences within the open reading frame of the arabinose binding protein domain such that the fluorescent moieties are expressed and displayed from a location within the nanosensor rather than at the termini. Such sensors are generally described in U.S. Application Ser. No. 60/658,141, which is herein incorporated by reference in its entirety. It will also be possible to insert a binding sequence, such as an arabinose binding sequence, into a single fluorophore coding sequence, i.e. a sequence encoding a GFP, YFP, CFP, BFP, etc., rather than between tandem molecules. According to the disclosures of U.S. Pat. No. 6,469,154 and U.S. Pat. No. 6,783,958, each of which is incorporated herein by reference in their entirety, such sensors respond by producing detectable changes within the protein that influence the activity of the fluorophore.

The present invention also encompasses isolated nanosensor molecules, including nanosensors for detecting levels of any metabolite, for example, pentose or hexose substrate, having the properties described herein, for instance, arabinose binding fluorescent indicators constructed using arabinose binding proteins, such as AraF. Such polypeptides may be recombinantly expressed using the nucleic acid constructs described herein, or produced by chemically coupling some or all of the component domains.

In one embodiment of the invention, the invention encompasses nanosensors for monitoring changes in intracellular levels of pentose and hexose substrates, which are both major components of plant cell walls. In yet another embodiment, the invention encompasses nanosensors for monitoring changes in intracellular levels of arabinose, including L-arabinose, D-arabinose, or D/L-arabinose, in a cell. According to one example, the nucleic acids and proteins of the present invention are useful for detecting L-arabinose binding and measuring changes in the levels of L-arabinose both in vitro and in a plant or an animal, and in particular in a bacterial cell.

The expressed nanosensors of the present invention can optionally be produced in and/or isolated from a transcription-translation system or from a recombinant cell, by biochemical and/or immunological purification methods known in the art. The polypeptides of the invention can be introduced into a lipid bilayer, such as a cellular membrane extract, or an artificial lipid bilayer or nanoparticle.

Nanosensors Based on Bioluminescence Resonance Energy Transfer

In one aspect of the invention, bioluminescence resonance energy transfer (BRET) may be used to monitor changes in ligand concentrations within a cell. In general, BRET technology is based on the transfer of resonant energy from a bioluminescent donor protein to a fluorescent acceptor protein. In one aspect of the invention, BRET may involve the transfer of resonant energy from a bioluminescent donor protein to a fluorescent acceptor protein using, for example, Renilla luciferase (Rluc) as the donor and a mutant of the Green Fluorescent Protein (GFP) as the acceptor molecule. The BRET technology is therefore analogous to fluorescence resonance energy transfer (FRET) as described herein, however BRET does not require an excitation light source.

In another aspect of the invention, a bioluminescent luciferase may be genetically fused to a candidate protein, and a green fluorescent protein mutant fused to another protein of interest. A ligand of interest may be known to be involved in cellular interactions between the two fusion proteins, for instance, the ligand may induce protein-protein interactions between the two fusion proteins. Increasing intracellular concentrations of the ligand can therefore bring the luciferase and the green fluorescent protein close enough for resonance energy transfer to occur, thus changing the color of the bioluminescent emission to indicate corresponding changes in ligand concentrations. BRET may also be useful for detecting the effect of a particular ligand on protein interactions within native cells, such as with integral membrane proteins or proteins targeted to specific organelles.

According to another aspect of the invention, bioluminescence resonance energy transfer may be used in assays to examine the effect of changes in ligand concentrations on activation of one or more receptors in intact living cells, such as G-protein coupled receptor activation in intact cells. Such receptors may be involved in one or more aspects of cellular homeostatis, uptake and/or efflux of one or more molecules into the cell, and/or aspects of cellular metabolism or other biochemical events and pathways within the cell. BRET may also be used, in accordance with the present invention, to continuously monitor the effects of changes in ligand concentrations on intracellular signaling events in live cells.

Devices for Measuring and Analyzing RET

The present invention also contemplates systems and devices for measuring and analyzing changes in resonance energy transfer (RET) which includes, but is not limited to, FRET, BRET and CRET, by measuring energy emission intensities, as described in further detail herein. In one example, a device for on-line measurement of changes in intracellular ligand concentrations in a cell is provided, in which the device includes a monitoring component operable for monitoring a living cell expressing a ligand binding fluorescent indicator, and an analyzing component operable for analyzing a change in energy emission, wherein the change in energy emission may indicate, for instance, a change in level of a ligand in the cell, or a change in utilization or metabolism of a ligand in the cell.

In one aspect of the invention, a system is provided for online monitoring of energy emission sensor output in cell cultures, fermentation media, or from any type of living cell whether in solution, or on a solid or semisolid substrate. The system may, for example, include light sources, selective optical filters, and recording devices that quantitatively measure photons. In general, the invention encompasses systems for real-time, online measurement of changes in resonance energy transfer in any type of cell transformed or transfected with a nanosensor of the present invention. The present invention thus provides systems that may be used for detecting and analyzing changes in the level or concentration of any intracellular ligand, including any type of pentose or hexose substrate, in living cells in general and in bacterial cells in particular.

In another aspect, the invention provides devices for the detection of intracellular ligand or metabolite concentrations in a bacterial cell culture, comprising a light source, a filter specific for excitation of an acceptor fluorophore, a filter specific for the excitation of a donor fluorophore, a filter specific for energy emission from the acceptor fluorophore, a filter specific for energy emission from the donor fluorophore, and a detector. In one aspect, the detector comprises at least two emission energy detectors such as photo multiplier detectors or charge-coupled device sensors; and a processor, which processes the signals from the detectors. Alternatively, devices can be constructed with components that record the emission spectrum of the donor and the acceptor moieties. The components may, for instance, include an illumination slit and a grating that disperses the light and some sort of emission energy detector, for instance a CCD chip that collects the intensities of the dispersed light of defined wavelengths. In another setup, the components may consist of a polychromator coupled to a diode array detector. In another aspect, the processor is operable for calculating a ratio of light intensity values, wherein the ratio comprises a ratio of energy emission values detected from the excitation of the donor fluorophore and the energy emission from the acceptor fluorophore. The light source may, for instance, include a LED, a xenon lamp, or a laser.

The term “monitoring component” as described herein, includes any suitable component of a system or device for monitoring and detecting a change in energy emission, including for instance, a system that employs fluorescent spectroscopy for measuring fluorescence emission.

In one embodiment, a system is operable for online measurement of FRET by recording fluorescence energy emission intensities at selected wavelengths after selective excitation of a FRET donor fluorophore. After excitation of the donor fluorophore, energy is transmitted to the FRET acceptor fluorophore in a non-radiative manner and emitted by the acceptor. The energy emission intensity of the FRET acceptor after its selective excitation is recorded to monitor the effect of the intracellular environment on the spectral properties of the fluorophores. It is to be understood that changes in RET may be measured using a variety of suitable techniques known in the art. One example is a spectrofluorometric system for online measurement of FRET. Another example is a system for detecting and analyzing changes in bioluminescence resonance energy transfer.

In one embodiment, the invention contemplates the use of a spectrofluorometric system for on-line measurement of intracellular ligand or metabolite concentrations in a living cell. Fluorescent spectroscopy may be used, for instance, for monitoring a sample containing cells transfected or transformed with a FRET nanosensor, for instance transfected mammalian cells or transformed bacterial cells. The sample may be placed in a holder, and the sample may be in the form of a liquid solution or suspension, or a solid on a substrate, such as a batch of living cells retained or immobilized on a filter paper.

A fluorescent spectrofluorometer, in accordance with the present invention, will typically include an excitation source for excitation of the donor and acceptor fluorophores, for instance, by means of a pulsed excitation source such as a pulsed dye laser excited by a nitrogen laser or by a nitrogen laser alone. A sample of cells transfected or transformed with a FRET nanosensor may be exposed to radiation of known spectral distribution, characteristically a restricted bandwidth of light. In one instance, the distribution of excitation radiation is typically within, or alternatively approximately within, the excitation spectrum of the donor and acceptor fluorophores in the FRET nanosensor.

The spectrofluorometer will typically also include a fluorescent detection system, for instance, a detection means utilizing a photomultiplier, which is typically spaced apart from the excitation source and positioned to receive fluorescent radiation emission from the donor and acceptor fluorophores in response to the excitation pulses. In one embodiment, the fluorescence detection system is gated on only after the fluorescence of ambient substances, which cause deleterious competing fluorescence, has substantially decayed.

Determining a change in FRET may include measuring light emitted from the acceptor fluorescent protein moiety after selective excitation of a FRET donor fluorophore. Alternatively, determining a change in FRET may include measuring light emitted from the donor fluorescent protein moiety, measuring light emitted from the acceptor fluorescent protein moiety, and calculating a ratio of the light emitted from the donor fluorescent protein moiety and the light emitted from the acceptor fluorescent protein moiety. The step of determining FRET may also comprise measuring the excited state lifetime of the donor moiety or anisotropy changes (Squire A, Verveer P J, Rocks O, Bastiaens P I. J Struct Biol. 2004 July; 147(1):62-9. Red-edge anisotropy microscopy enables dynamic imaging of homo-FRET between green fluorescent proteins in cells.). Such methods are known in the art and described in U.S. Pat. No. 6,197,928, which is incorporated by reference in its entirety.

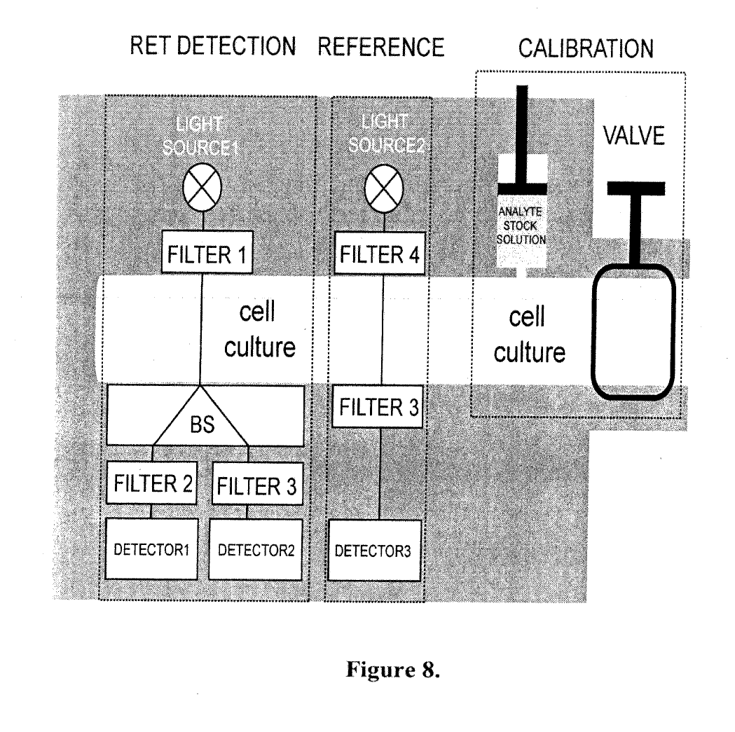

The systems of the present invention also provide for detection and analysis of fluorescent emission ratios by use of a fluorescence emission ratio analyzer. The operation of one exemplary fluorescent detection system is described with reference to FIG. 8, in which a FRET nanosensor response in bacterial cells can be monitored using a fluorescence emission ratio analyzer. As depicted schematically in FIG. 8, the device is mounted inside a fermenter, or alternatively fermenter culture is passed through it continuously. The device depicted in FIG. 8 consists of a light source whose light passes through two filters. Light sources can include, for instance, light-emitting diodes (LEDs) or other light emitters such as xenon lamps or lasers. Filter 1 is specific for only allowing transmission of excitation energy for excitation of a FRET acceptor fluorescent protein, such as Venus. Filter 2 is specific for only allowing transmission of excitation energy for excitation of a FRET donor fluorescent protein, such as eCFP. Filter 3 is specific for only allowing transmission of FRET acceptor emission light, in this case Venus emission. Filter 4 is specific for only allowing transmission of FRET donor emission light, in this case eCFP emission. Light recorded by a detector which measures the light intensity (here abbreviated PMD for photo multiplier detector) can be constructed in various ways, for instance, a simple light sensitive diode or CCD chip. The light detected by PMD1 serves as a control for the quality of the fluorescence of the FRET acceptor fluorescent protein, in this case Venus. The PMD2/PMD3 light ratio gives the output of the sensor. The fluorescent detection system can also be equipped with an injector to calibrate the measurement cell with external ligands, e.g. arabinose at different concentrations.

When bioluminescence resonance energy transfer (BRET) is employed for the detection of changes in, for instance, intracellular ligand concentrations, BRET may involve the transfer of resonant energy from a bioluminescent donor protein to a fluorescent acceptor protein. In one example, BRET may use Renilla luciferase (Rluc) as the donor and a mutant of the Green Fluorescent Protein (GFP) as the acceptor molecule. The BRET technology is therefore analogous to fluorescence resonance energy transfer (FRET) as described herein, however BRET does not require an excitation light source. Instead, when BRET is employed, the invention contemplates devices that can continuously detect changes in intracellular ligand concentrations by monitoring for changes in emission intensities of the bioluminescence donor protein and the acceptor protein.

The output detected by the detector, including the emission spectrum following excitation by an excitation source, and as described above in one example with reference to FIG. 8, can be further analyzed by any suitable analyzing component. The “analyzing component” as described herein, includes any suitable device for analyzing a change in energy emission ratios, for instance, a fluorescence emission ratio analyzer, or other component that analyzes a change in energy emission due to resonance energy transfer.

The systems of the present invention may also include a processing system for further processing and analyzing sensor output, including changes in energy emission that are detected and analyzed by a fluorescence emission ratio analyzer. An exemplary processing system may utilize a memory device, for instance, a DDR SDRAM or other RAM device having unidirectional row logic, and routing and power bussing. The processing system may include one or more processors coupled to a local bus, multiple memory controllers and/or multiple primary bus bridges. In addition, the memory controller may be coupled to one or more memory buses, and each memory bus may accept memory components which include at least one memory device. The memory components may also include a memory card or a memory module. Examples of memory modules include single inline memory modules (SIMMs) and dual inline memory modules (DIMMs). The memory controller may also be coupled to a cache memory. Any suitable processing architecture may be employed that is especially suitable for a general purpose computer, such as a personal computer or a workstation, for further processing and analyzing sensor output, including changes in energy emission that are detected and analyzed by a fluorescence emission ratio analyzer. Sensor output data may also be stored, transmitted, or otherwise utilized as necessary or desired, and it is to be understood that the systems of the present invention may be operably connected to data storage devices and other peripheral devices such as servers and printers, and it should be recognized that well known modifications can be made to configure the processing system to become more suitable for use in a variety of applications. The modifications may include, for example, addition of specialized devices or circuits, and/or integration of a plurality of devices.

The processes and devices described above illustrate exemplary methods and typical devices of many that could be used and produced. The above description illustrates embodiments, which achieve the objects, features, and advantages of the present invention. However, it is not intended that the present invention be strictly limited to the above-described and illustrated embodiments. One skilled in the art will recognize that, while certain examples have been described herein, other suitable methods, systems and devices may also be used to achieve the objects of the present invention.

Utilities

The biosensors of the present invention may be useful for a wide range of applications. In one embodiment, cells transformed or transfected with the nanosensors of the present invention can be analyzed for their steady state levels or changes in levels of a molecule, for example the levels of arabinose or maltose or any pentose or hexose substrate, in one or more intracellular compartments.

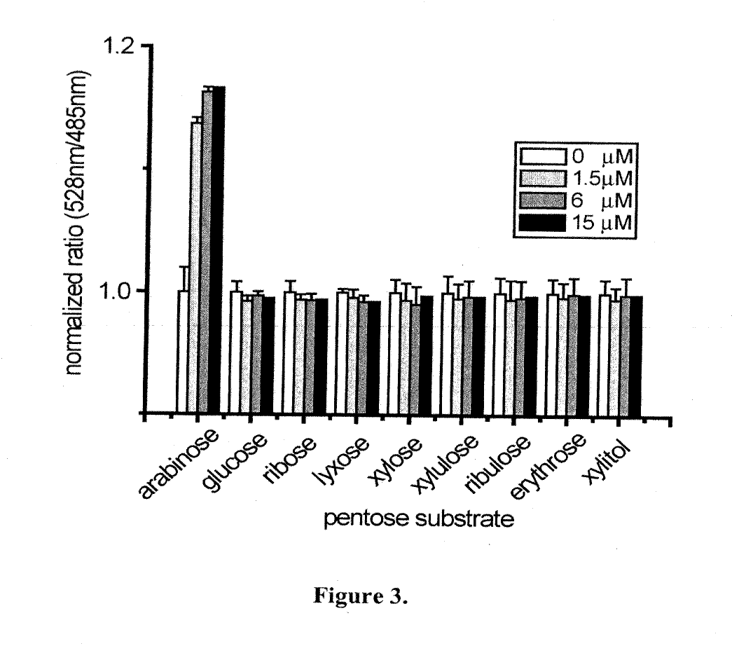

The amount of any molecule, such as a ligand, a substrate, a metabolite, in a sample of cells can be determined by detecting or monitoring and analyzing a change in RET. The substrate or metabolite may for instance be a pentose or hexose substrate. Examples of substrates include, but are not limited to, arabinose, glucose, ribose, xylose, xylulose, ribulose, erythrose, and xylitol. In one aspect of the invention, the nanosensor is first introduced into the sample of cells by any suitable transfection or transformation assay, such that the cells express the nanosensor. The cells may then be incubated, and after an appropriate interval of time, various concentrations of the pentose or hexose substrate are added to the sample of cells. Uptake of the substrate in the cell can be monitored and analyzed by recording the energy emission ratios. Changes in the metabolism or intracellular utilization of the pentose or hexose substrate can also be determined by monitoring the energy emission ratios. Such transformed or transfected cells may also be analyzed for changes in RET, according to the methods described herein, wherein changes in RET are analyzed for correlations with changes in steady state levels of other small molecules or metabolites within the cell. As depicted in FIG. 3, the emission ratio of a FRET nanosensor may be analyzed in response to different concentrations of different pentose substrates. The amount of substrate in the sample can be quantified for example by using a calibration curve established by titration. For establishing such calibration curves, it is desired that the same experimental conditions be applied as present during the effective measurement.

The present invention provides a method of detecting and/or monitoring metabolic flux in whole-cells. As described herein, the term “metabolic flux” includes any change that occurs in the metabolic state of a cell, microbe or microorganism. Furthermore, the term “metabolic state” broadly encompasses the overall state of metabolism within the cell, e.g., a cell of a microbe or microorganism, including all the accompanying biochemical processes that occur within the cell that accompany cellular activities, such as energy utilization, and in general any and all catabolic and anabolic processes within the cell, including but not limited to, catabolic and anabolic processes involved in growth, replication, maintaining structural integrity, and responses to environmental stimuli.

It is known for a long time that metabolism is highly regulated at all levels, including transcriptional, posttranslational, and allosteric controls. Flux through a metabolic or signaling pathway is determined by the activity of its individual components. Fluxomics aims to define the genes involved in regulation by following the flux. Flux and fluxomics are discussed in detail by Wiechart et al. (Current Opinion in Plant Biology, 2007, 10: 323-330), which is incorporated by reference in its entirety. Fluxes may be monitored by pulse labeling of the organism or cell with a tracer, such as ¹³C, followed by mass spectrometric analysis of the partitioning of label into different compounds. This is an efficient tool to study flux and allows the comparison of the effect of mutation on flux. Fluxes may also be monitored using nanosensors.

The whole-cells could be prokaryotic or eukaryotic cells. The cells could be bacterial cells, yeast cells, mammalian cells, or plant cells. The nucleic acid encoding nanosensors are introduced into the cells and the cells are cultivated under conditions that allow expression of the nanosensors. The method of the present invention involves measuring at least one metabolic parameter of the cell by monitoring the cell culture expressing the nanosensor in real time and analyzing the data. Alternatively, the analysis may involve comparing the data to determine if the measured parameter differs from a comparable measurement in a control cell. A control cell may be a normal cell or a cell that has not been modified to have a specific phenotype.

In the present invention, the cells are monitored in a real time or on-line time frame. In one aspect, a plurality of cells, such as a cell culture, is monitored in real time or on-line. In another aspect, a plurality of metabolic parameters is monitored in real time or on-line. The methods of the invention may be used to determine how to manipulate a cellular process, for example fermentation, by determining how to change the substrate supply, the temperature, use of inducers, etc. to control the physiological state of the cells to proceed in a desirable direction. As an example, the present invention will provide information for improving crop yields in plant for food and biofuel production.

The present invention also provides for monitoring intracellular metabolite concentrations in a bacterial cell culture, such as during a fermentation process. The described nanosensors thus have applications for any fermentation process, for instance, in the food, pharmaceutical, and biofuel industries. The present invention also provides for examining metabolic processes involved in ethanol production, for instance, ethanol production from sugar mixtures containing glucose and arabinose. For instance, an arabinose nanosensor may be used to detect changes in arabinose utilization during ethanol production in engineered cells.

As used herein, the term “fermentation” is to be understood to include any process, for instance in the context of industrial fermentation, that involves the breakdown of organic substances and re-assembly into other substances. Fermenter culture in industrial capacity often refers to highly oxygenated and aerobic growth conditions, whereas fermentation in the biochemical context is an anaerobic process.

For efficient operation of any fermentation process, the amount of substrate must be maintained at a certain level. Accordingly, the fermenter substrate concentration must be monitored. Starch is used as a cheap source of substrate for fermentation processes and is broken down by microorganisms into glucose before consumption, which may result in the accumulation of glucose during the fermentation process. However, the accumulation of glucose above a certain concentration in any starch hydrolysis process may inhibit the action of starch hydrolysing enzymes. Thus, it is necessary to monitor the concentration of both glucose and starch (total sugars) during the fermentation process. As an example, the nanosensors of the present invention may be used to monitor both glucose and the total sugar during the fermentation process. The nanosensors may be used to monitor other sugars and metabolites involved in the fermentation process. The nanosensors can be used for continuous monitoring of metabolites in real time or on-line time frame and will provide immediate information.

The nanosensors of the present invention may be used to monitor, for instance, the accumulation of metabolites, which can indicate obstruction in or repression of metabolic pathways. Alternatively, reduced intracellular metabolite levels may indicate a lack of external supplies. A change in energy emission may indicate a change in level or concentration of an intracellular ligand during a fermentation process. The ligand may include, for instance, a sugar, amino acid, peptide, organic acid, metals or ion, oxide, hydroxide or conjugate thereof, inorganic ion, (poly) amine and vitamin. As depicted in FIG. 8, a FRET nanosensor response in bacterial cells can be monitored using a simple fluorescence emission ratio analyzer.

As used herein, the term “biofuel” broadly includes any solid, liquid, or gas fuel that consists of, or is derived from, biomass, for instance, any suitable biological material which can be used as fuel or for industrial production, including carbon-based materials. Examples of biofuels, in accordance with the present invention, are fuels derived from biomass that are generated by microbes or microorganisms engineered for the production of such fuels.

The present invention also provides for expression of fluorescent nanosensors in cells for on-line monitoring of metabolic state. The cells may be obtained from any source including, for instance, prokaryotic cells, such as E. coli or other bacteria, or eukaryotic cells, such as yeast cells, animal cells or plant cells. Any suitable microorganism may be used to express a fluorescent nanosensor, such that the microorganism itself is operable as a whole-cell biosensor for online detection of specific molecule levels. The invention also contemplates the monitoring of metabolic processes under aerobic or anaerobic conditions.

The present invention also provides for real-time detection and monitoring of metabolite levels in cells, including monitoring of metabolic flux in cells. In one embodiment of the invention, methods of determining metabolic flux in a living cell are provided, including methods of monitoring a living cell expressing a ligand binding fluorescent indicator as described herein, and determining a change in RET which correlates with a change in the metabolic flux within the cell. In another embodiment, the invention contemplates determining metabolic flux in microbial cells that are engineered to produce biofuels or other biotechnological products.

In one aspect of the invention, methods of determining metabolic flux in a living cell are provided for real-time, online and/or continuous monitoring of the metabolic state of a cell during a fermentation process. The inventors of the present invention have surprisingly found that the RET nanosensors may be used for monitoring the specific detection of the pentose arabinose and demonstrate that such sensors can successfully be used to monitor flux (with the example of arabinose and maltose) in bacteria, specifically E. coli using simple fluorescence spectroscopy. Changes in metabolic flux may also be monitored in response to changes in one or more extracellular and/or intracellular conditions, e.g., changes in light, sugar supply, humidity, addition of one or more exogenous compounds, and/or other changes within the cellular microenvironment that affect cellular metabolism and utilization of small molecules or metabolites within the cell.

In one embodiment, the invention comprises a method of detecting changes in the level of a metabolite in a sample of cells, comprising monitoring a cell expressing a nanosensor as described herein, and detecting a change in FRET between a donor fluorescent protein moiety and an acceptor fluorescent protein moiety, wherein a change in FRET between said donor moiety and said acceptor moiety indicates a change in the level of the metabolite in the sample of cells. According to one embodiment, the inventors have surprisingly and unexpectedly discovered that the FRET sensors of the invention can be used for monitoring intracellular metabolite concentrations in any bacterial cell culture.

Methods for detecting metabolite levels as disclosed herein may also be used to screen and identify compounds that may be used to modulate metabolite concentrations and activities relating to metabolite changes. In one embodiment, among others, the invention comprises a method of identifying a compound that modulates metabolite binding or levels comprising (a) contacting a mixture comprising a cell expressing a nanosensor as disclosed herein and a sample of cells with one or more test compounds, and (b) determining FRET between said donor fluorescent domain and said acceptor fluorescent domain following said contacting, wherein increased or decreased FRET following said contacting indicates that said test compound is a compound that modulates metabolite activity or metabolite levels. The term “modulate” in this embodiment means that such compounds may increase or decrease metabolite activity, or may affect activities, i.e., cell functions or signaling cascades, that affect metabolite levels. Compounds that increase or decrease metabolite activity may be targets for therapeutic intervention and treatment of disorders associated with aberrant metabolite activity, or with aberrant cell metabolism or signal transduction, as described above. Other compounds that increase or decrease metabolite activity or metabolite levels associated with cellular functions may be developed into therapeutic products for the treatment of disorders associated with the activity.

According to one example, detection of a change in RET may be used to detect changes in arabinose concentrations within a cell. Arabinose is an aldopentose (a monosaccharide) and is found in nature as a component of biopolymers such as pectin and hemicellulose. Arabinose can be metabolized by microorganisms as a carbon source. Arabinose may be made synthetically from glucose, and is often used in culture media in bacteriology. Another important saccharide is maltose which is a disaccharide formed from two units of glucose joined with an α(1→4) linkage. Among its many uses, maltose is also commonly used in culture media in bacteriology.

The nanosensors of the present invention will also help elucidate the mechanisms for synthesis of metabolites in cells, and in particular in bacteria. Such sensors will also provide a unique opportunity to measure fluxes in living cells, including uptake or influx of a ligand or substrate in to a cell, the subcellular distribution and/or compartmentalization of the ligand within the cell, and efflux of the ligand or substrate form the cell. Such sensors will also have value as tools to identify functions such as ligand or substrate utilization in cells, and to identify the regulators and the signaling cascades controlling cellular homeostasis. Thus, the sensor can be used to characterize cellular activities including, for instance, uptake and release of ligands, and also intracellular compartmentation. The nanosensors of the present invention may thus be used to monitor intracellular levels of small molecules, for instance, arabinose or maltose, in cells, or for real-time monitoring of metabolite levels in cells. It is also to be understood that the applications and utilities as described herein will also apply for the monitoring of intracellular ligand concentrations under either aerobic or anaerobic conditions, and also under any other experimental conditions including, for instance, changes in the cellular medium such as changes in pH, temperature, ionic conditions, or any other factors that may affect ligand binding and, consequently, energy emission due to resonance energy transfer.

The nanosensors of the present invention can also be used to characterize the link between synthesis and growth and yield of specific cells and tissues under a variety of different environmental conditions which can be affected by such factors as carbon dioxide utilization, oxygen sensitivity, temperature-dependent growth responsiveness and expression of endogenous genes responsive to intracellular factors or external factors outside the cell. The sensors will be also useful to study the biochemical pathways, for instance, to determine arabinose pathways in microorganisms. It can be used as a tool to develop new chemicals that positively or negatively affect arabinose synthesis in high throughput screens. The arabinose sensors of the present invention are excellent tools for drug discovery and screening. Levels of metabolites, such as arabinose, may be measured in real time in response to chemicals, metabolic events, transport steps and signaling processes. The sensors can also be used to monitor aberrant changes in metabolite levels in cells and thus have utility in helping to diagnose diseases associated with aberrant metabolism. For instance, the nanosensors may provide tools to investigate the underlying defects and to develop cures for diseases associated with aberrant metabolism.

In accordance with the methods and systems described herein, the present invention also contemplates that a cell may be transfected or transformed with more than one nanosensor, wherein each nanosensor includes a ligand-sensing domain that is specific for a different ligand. Such a cell may be present, for instance, in a sample of bacterial cell culture or a sample from a fermentation process, and may be scanned with a combined excitation/detection means for monitoring and analyzing changes in energy emission due to resonance energy transfer, as described herein. Such a system may be of particular utility in automated or semi-automated high throughput screening of cell samples, for instance, in monitoring for simultaneous changes in intracellular concentrations of different ligands. One skilled in the art will recognize that the simultaneous monitoring of more than one nanosensor may provide different output, for example, an increase in the intracellular concentration of one ligand concurrent with a decrease in the intracellular concentration of another ligand, or simultaneous increases in both ligand concentrations, no change in the steady state of one or both ligand concentrations.

The following examples are provided to describe and illustrate the present invention. As such, they should not be construed to limit the scope of the invention. Those in the art will well appreciate that many other embodiments also fall within the scope of the invention, as it is described hereinabove and in the claims.

### EXAMPLES

### Example 1

### Construction of an Arabinose FRET Nanosensor

The araF gene (EMBL: K00420) was amplified from Escherichia coli K12 genomic DNA using homologous primers, resulting in a PCR product that consisted of araF without stopcodon flanked by phage lambda attB recombination sites. The araF gene was fused between eCFP and Venus coding sequences of pGW1 through pDONR (Invitrogen) using LR and BP recombinases (Invitrogen), respectively. The final construct was denoted pGW1araF.Ec and the correct sequence of the araF gene was verified by DNA sequencing (Sequetech). The gene product encoded on pGW1araF.Ec was denoted FLIParaF.Ec-200n.

### Example 2

### In Vitro Characterization of Arabinose FRET Nanosensor

FLIPW constructs were harbored in E. coli BL21(DE3) gold and sensor proteins were produced and purified as described previously (Fehr, M., Frommer, W. B. and Lalonde, S., 2002, Visualization of maltose uptake in living yeast cells by fluorescent nanosensors. Proc. Natl. Acad. Sci. U.S.A. 99, 9846-9851). Purified sensor was added to a dilution series of ligand in 20 mM MOPS pH 7.0 in the range of 10⁻⁴ to 10⁻⁹ M and analyzed in a monochromator microplate reader (Safire, Tecan, Austria; eCFP excitation 433/12 nm, eCFP emission 485/12 and Venus emission 528/12 nm). eCFP shows two emission peaks at 476 nm and 501 nm (LaMorte, V. J., Zoumi, A. and Tromberg, B. J., 2003, Spectroscopic approach for monitoring two-photon excited fluorescence resonance energy transfer from homodimers at the subcellular level. J. Biomed. Opt. 8, 357-361). The eCFP emission used for the ratio calculation was determined at 485 nm. Protein was diluted to give Venus/eYFP readouts of 20,000 to 30,000 at a manual gain between 70-75. By using the change in FRET ratio upon binding of ligand, affinity constants (K_{d}) were determined by fitting the titration curves to a single-site-binding isotherm:

R=Rₐₚₒ+(Rₛₐₜ−Rₐₚₒ)·(n·[L])/(K_{d}+[L])

with [L], ligand concentration; n, number of equal binding sites; R, ratio; Rₐₚₒ, ratio in the absence of ligand; and Rₛₐₜ, ratio at saturation with ligand. Three independent protein preparations were analyzed and each protein preparation was analyzed in triplicate.

### Example 3

### Monitoring of Intracellular Small Molecule Levels Using Fret Nanosensors

E. coli BL21(DE3) cells were transformed with pGW1FaraF.Ec or p3367, which encode an arabinose FLIP nanosensor with a K_{d} of 200 nM and a maltose FLIP nanosensor with a K_{d} of 37 μM, respectively. Cultures were incubated in LB in baffled Erlenmeyer flasks for 48 hrs in the dark at room temperature while shaking, and stored at least 16 hrs at 4° C. 5 ml of culture was spun down, washed in 10 ml M9 minimal salts medium, spun down, and resuspended in 9 ml M9 medium. Cells were dispended in a microplate at 90 μl per well. In a microplate fluorescence spectrophotometer the sensor output was monitored with 150 sec intervals by exciting eCFP at 433/6 nm and recording eCFP and Venus emissions at 485/6 nm and 528/6 nm, respectively. After 13 min incubation, 10 μl of various concentrations arabinose or maltose in M9 medium were added to the cells and the fluorophore emissions were recorded for another 45 min. Venus/eCFP ratios were normalized against cells to which 10 μl M9 medium had been added. To monitor accumulation rates, the injection module of a Tecan Infinite M200 (Tecan, Australia) was used.

### Example 4

### In Vitro Characterization of an Arabinose FRET Nanosensor

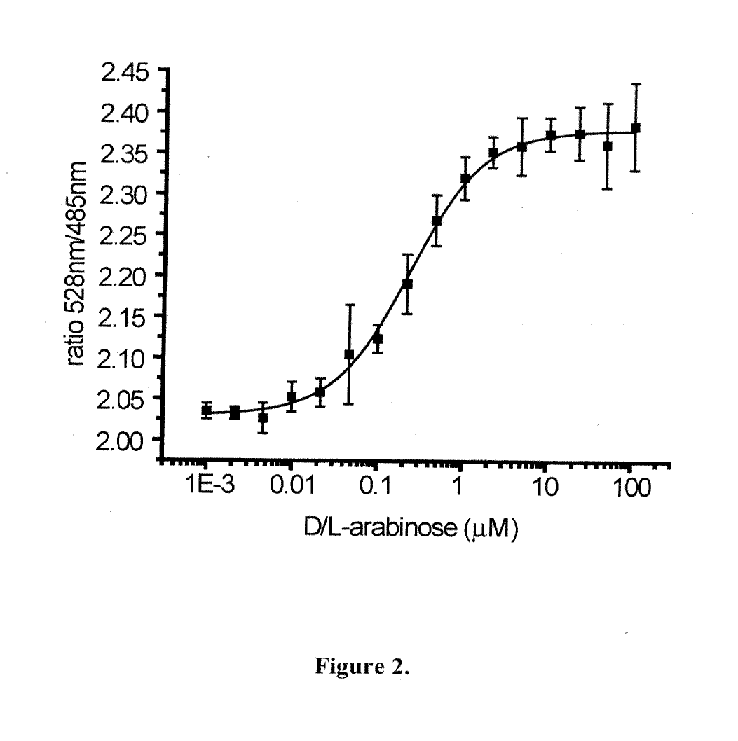

A FRET nanosensor for detection of L-arabinose was constructed by the fusion of E. coli K12araF between eCFP and Venus coding sequences. Production of the translated fusion product FLIParaF.Ec in E. coli was readily detected by recording the emission spectrum of the eCFP-Venus FRET signal in whole cell cultures. When eCFP was excited, significant energy transfer to Venus was detected, resulting in a Venus/eCFP emission ratio of 2. Addition of D/L-arabinose increased FRET efficiency of the purified protein, visible as a decrease in eCFP emission intensity and a concomitant increase in Venus fluorescence intensity, resulting in a ˜15% increase in the emission ratio (FIG. 2). FLIParaF.Ec bound D/L-arabinose with an apparent K_{d} of 230±18 nM. AraF binds L-arabinose with an affinity of 98±33 nM (Miller, D. M., 3rd, Olson, J. S., Pflugrath, J. W. and Quiocho, F. A., 1983, Rates of ligand binding to periplasmic proteins involved in bacterial transport and chemotaxis. J Biol Chem 258(22), 13665-72). Assuming that the D/L-arabinose mixture consists of equal parts D and L isomer, the affinity of the arabinose FRET nanosensor for L-arabinose is equal to that of unmodified AraF as measured by tryptophan fluorescence sprectroscopy (Miller, D. M., 3rd, Olson, J. S., Pflugrath, J. W. and Quiocho, F. A., 1983, Rates of ligand binding to periplasmic proteins involved in bacterial transport and chemotaxis. J Biol Chem 258(22), 13665-72). FLIParaF.Ec-200n specifically interacts with arabinose, as several other pentose substrates failed to induce a FRET ratio change (FIG. 3).

### Example 5

### Monitoring Intracellular Arabinose Levels in E. coli

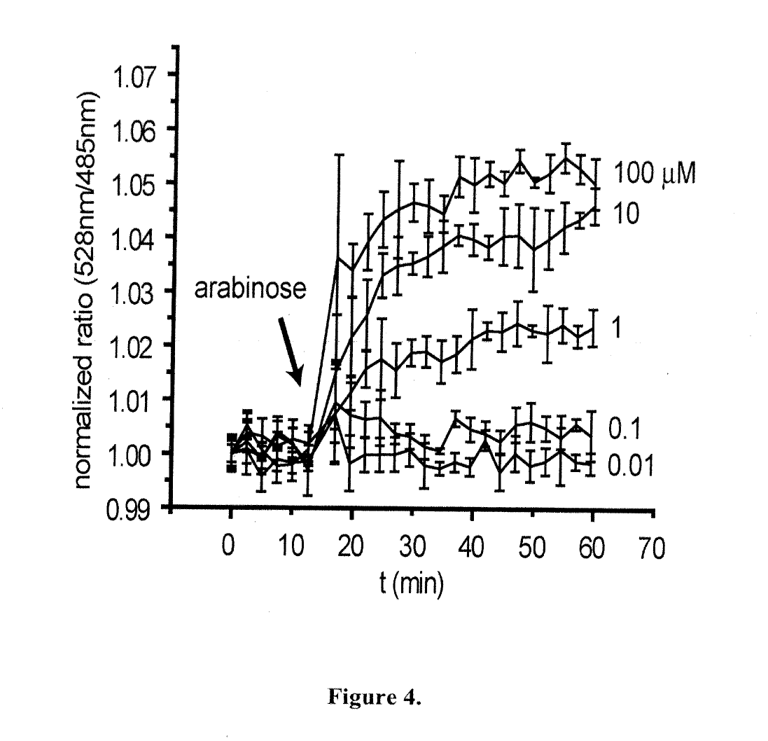

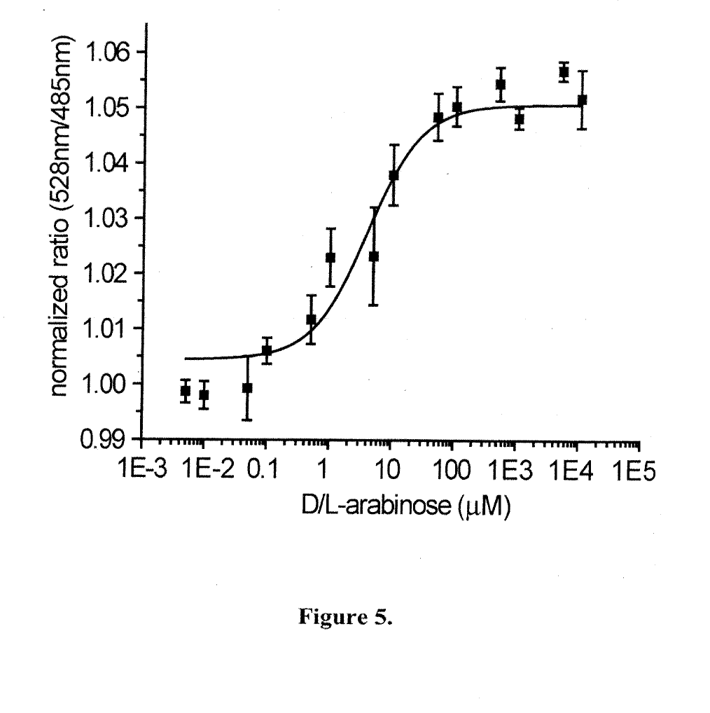

FLIParaF.Ec-200n was produced in E. coli BL21(DE3). Different concentrations of arabinose were added to the E. coli cell cultures and arabinose uptake was monitored by recording the FLIParaF.Ec-200n emission ratios (FIG. 4). Initial monitoring gave a stable baseline. Upon addition of arabinose the emission ratio were stable between 0-0.1 μM, increased between 0.1 μM and 500 μM external arabinose, were high between 500 μM-10 mM and dropped slightly at external arabinose concentrations of 50 and 100 mM, the two highest concentrations used. The emission ratios after 50 min were plotted against the external arabinose concentration, which gave a curve, which showed that the intracellular arbinose concentration was 15-fold lower than outside the cells (FIG. 5).

### Example 6

### Construction of a Maltose Sensor

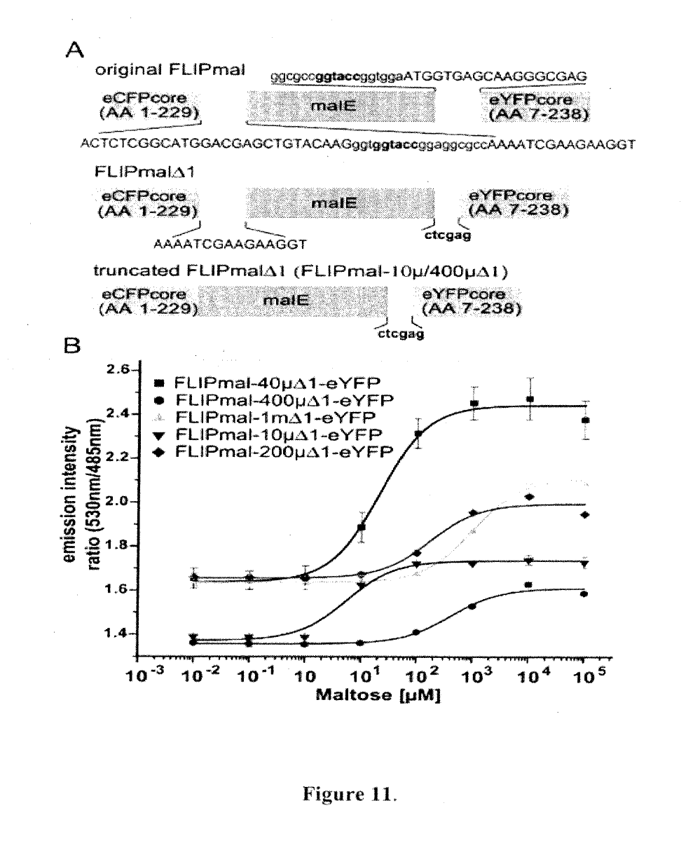

To test whether this simple detection system can be used for monitoring cytosolic maltose levels, and to test whether an increased sensitivity of the assay can be obtained with optimized FRET sensors, a set of improved maltose sensors was constructed by linker deletions and expressed in E. coli. A maltose sensor FLIPmal-25g had previously been constructed by fusing the E. coli MalE protein with eCFP and eYFP fluorescent proteins (Fehr et al. PNAS 99:9846-9851 (2002)). However, FLIPmal-25μ showed only a modest ratio change in the presence of maltose. Linker deletions have been used successfully to improve the ratio change of glucose FRET sensors (Deuschle et al. Protein Sci. 12: 2304-2314 (2005)), and the same strategy was applied here to the maltose sensor, resulting in a set of maltose sensors with K_{d}s ranging from the low to high micromolar range (FIG. 11). A maltose sensor was constructed by fusing the E. coli MalE protein with eCFP and Venus fluorescent proteins. The plasmid encoding the sensor was denoted p3367. The sensor displayed a maltose-dependent FRET change with an apparent K_{d} of 37 μM (FLIPmal-37μ (also FLIPmal-40μΔ1-V)).

### Example 7

### Monitoring Intracellular Maltose Levels in E. coli

FLIPmal-225μ, carrying the mutation W62A, was used as a base for generating improved FLIPmal sensors (Fehr et al. PNAS 99:9846-9851 (2002)). Seventy-five nucleotides (25 amino acids; FIG. 11A) of the linker regions were removed in FLIPmal-225μ by site-directed mutagenesis (Kunkel, PNAS 82: 488-492 (1985)) similar as previously done in the FLIPgluΔ13 series (Deuschle et al. Protein Sci. 12: 2304-2314 (2005)). This new FLIPmal sensor had a K_{d} for maltose of 200 μM and was denoted by FLIPmal-200 μΔ1-eYFP (FLIPmal-200 μΔ1-eYFP carries the mutation W62A). Affinity mutants of this sensor were created by structure-guided, site-directed mutagenesis of the binding pocket, yielding. FLIPmal-40μΔ1-eYFP (carrying the mutation W230A in addition to W62A) and FLIPmal-1mΔ1-eYFP (carrying the mutation Y155A in addition to W62A). In addition, two sensors were constructed in which the first five amino acids of malE were deleted, which earlier had been shown to generate a sensor with a K_{d} in the low micromolar range (Deuschle et al. Protein Sci. 12: 2304-2314 (2005)). This five-amino-acid deletion in the W62A background gave a sensor with a K_{d} of 400 μM (FLIPmal-400μΔ-eYFP). When W62A was reverted back to W62W, the affinity of the sensor was found to be 10 μM (FLIPmal-10μΔ1-eYFP). When the clones were sequenced, an additional mutation (N227A) was found in FLIPmal-40μΔ1-eYFP (FLIPmal-37μ), which probably occurred as an artifact during site-directed mutagenesis.

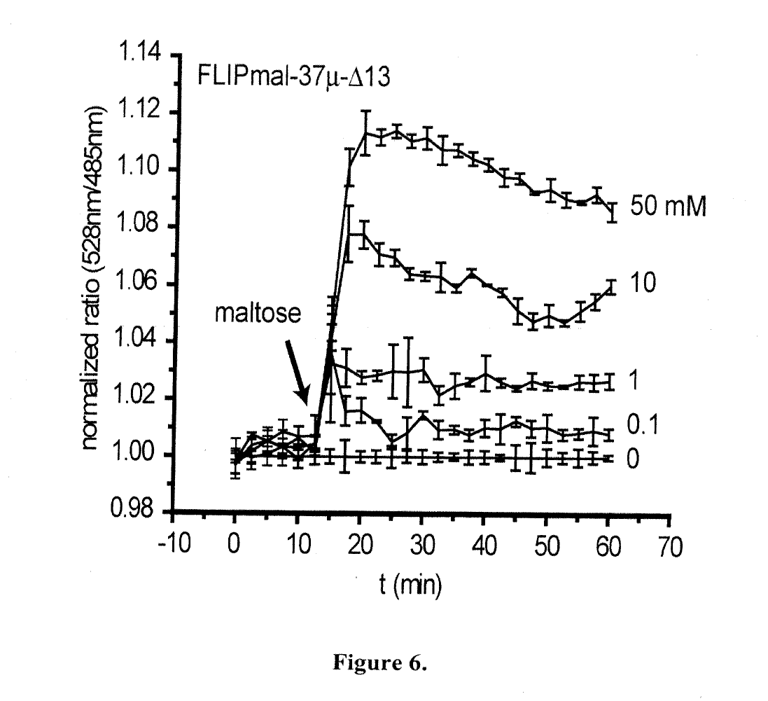

FLIPmal-37μ (also referred to as FLIPmal-40μΔ1-V) was produced in BL21(DE3). Different concentrations of maltose were added to the E. coli cell cultures and maltose uptake was monitored by recording the FLIPmal-37μ emission ratios (FIG. 4). Initial monitoring of emission ratios gave a stable baseline (FIG. 6). Upon addition of maltose the ratio increased in a concentration dependent manner: the higher the concentration, the larger the ratio. At 50 and 100 μM maltose the sensor responded immediately after addition and 3 min after addition the response dropped significantly. In the case of 50 μM the response dropped to baseline level. At higher maltose concentrations, the response of the sensor remained above the baseline level.

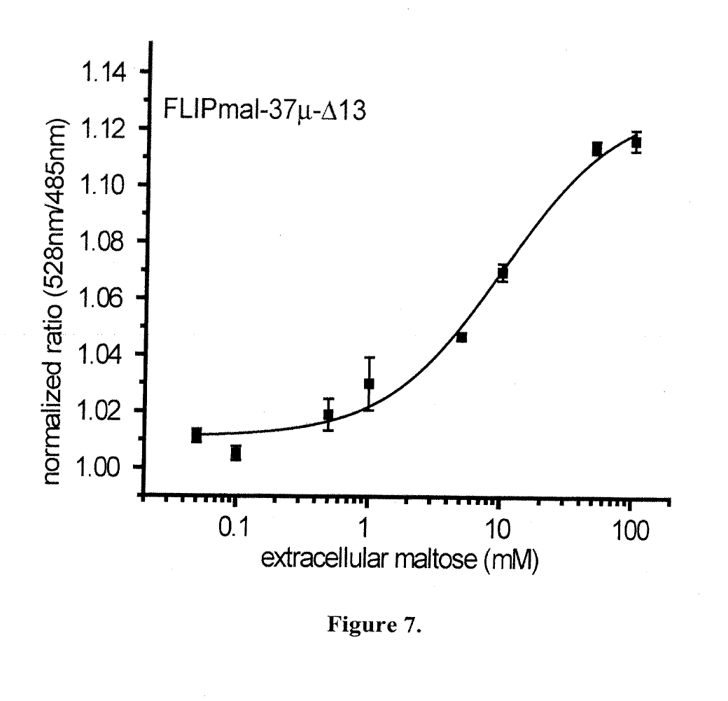

When the sensor responses at t=25 min are plotted against the external maltose concentration, a concentration dependent response is visible that can be fitted with a single site binding curve (FIG. 7). The apparent K_{d} derived from the curve is 10 mM, which is about 250-fold higher than the K_{d} of the sensor in vitro. Assuming the K_{d} of the sensor in the cytosol of E. coli is unchanged, this means that there exists a 250-fold gradient between outside and inside maltose levels.

### Example 8

### Fret Monitor for Online Detection of Sensor Output

The FRET nanosensor response in cell suspensions can be monitored using a simple fluorescence emission ratio analyzer (FIG. 8). The device is mounted inside a fermenter, or fermenter culture is passed through it continuously. It consists of a light source whose light passes through two filters. Light sources can be LEDs or other light emitters such as xenon lamps or lasers. Filter 1 is for specific excitation of FRET acceptor fluorescent protein, in this case Venus. Filter 2 is for excitation of FRET donor fluorescent protein, in this case eCFP. Filter 3 is specific for FRET acceptor emission light, in this case Venus emission. Filter 4 is specific for FRET donor emission light, in this case eCFP emission. Light recorded by a detector which measures the light intensity (here abbreviated PMD for photo multiplier detector) can be constructed in various ways, simple light sensitive diode, CCD chip, etc. PMD1 serves as a control for the quality of the fluorescence of the FRET acceptor fluorescent protein, in this case Venus. The PMD2/PMD3 light ratio gives the output of the sensor. The system can be equipped with an injector to calibrate the measurement cell with external ligands, e.g. arabinose at different concentrations.

### Example 9

### Monitoring Flux Changes Using a Glucose Nanosensor

A glucose nanosensor that has an affinity constant for glucose of 0.60 mM displays 10%, 20%, 80% and 90% saturation at 78 μM, 175 μM, 2.8 mM and 6.3 mM. The confidence range for calculation of intracellular glucose concentrations is determined by the quality of the recorded sensor response data. As a rule of thumb, data with standard deviations off ±2.5% and ±5% can be interpreted between 10% and 90% and between 20% and 80% saturation, respectively. Monitoring the sensor response in presence of alternating analyte concentrations yields information about transport and metabolism in the targeted subcellular compartment from (1) the intracellular accumulation rate upon addition of the analyte, which is composed of influx rate minus metabolism and export rates, (2) the elimination rate when the analyte is removed, which is composed of export plus metabolism minus influx, (3) the steady state level of the sensor response, in which influx equals metabolism and efflux, and (4) the time delay before a sensor response can be recorded, which represents differences in influx rates or metabolism.

All publications, patents and patent applications discussed herein are incorporated herein by reference. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed:

1. A method for detecting intracellular levels of a molecule in a living cell culture comprising expressing a nanosensor in living cells in culture, wherein the nanosensor comprises a donor moiety, an acceptor moiety, and a molecule binding site, and detecting a change in resonance energy transfer between the acceptor moiety and the donor moiety, thereby detecting the level of the molecule in the living cell culture.

2. The method of claim 1, wherein the molecule binding site is on the donor moiety.

3. The method of claim 1, wherein the resonance energy transfer is fluorescence resonance energy transfer, phosphorescence resonance energy transfer, chemiluminescence resonance energy transfer, or bioluminescence resonance energy transfer.

4. The method of claim 1, wherein the molecule is selected from the group consisting of sugars, amino acids, peptides, organic acids, metals or ions, oxides, hydroxides or conjugates thereof, inorganic ions, amines, polyamines and vitamins.

5. The method of claim 4, wherein the sugars are selected from the group consisting of arabinose, maltose, glucose, galactose, sucrose, trehalose, fructose, xylose, cellobiose and ribose.

6. The method of claim 1, wherein the living cell cultures comprise prokaryotic cells.

7. The method of claim 1, wherein the prokaryotic cells are bacteria or archaea.

8. The method of claim 1, wherein the intracellular levels of the molecule is detected in a real time or on-line time frame.

9. The method of claim 1, wherein detecting the intracellular levels of the molecule comprises calculating a ratio of energy emission values detected from the emission of the donor moiety and the emission from the acceptor moiety.

10. A method of monitoring the intracellular levels of a molecule in a living cell culture comprising expressing a nanosensor in living cells in culture, wherein the nanosensor comprises a donor moiety, an acceptor moiety, and a molecule binding site, detecting a change in resonance energy transfer between the acceptor moiety and the donor moiety, thereby monitoring the level of the molecule in the living cell culture.

11. The method of claim 10, wherein the molecule binding site is on the donor moiety.

12. The method of claim 10, wherein the resonance energy transfer is fluorescence energy transfer, phosphorescence energy transfer, chemiluminescence energy transfer or bioluminescence energy transfer.

13. The method of claim 10, wherein the molecule is selected from the group consisting of sugars, amino acids, peptides, organic acids, metals or ions, oxides, hydroxides or conjugates thereof, inorganic ions, amines, polyamines and vitamins.

14. The method of claim 13, wherein the sugars are selected from the group consisting of arabinose, maltose, glucose, galactose, sucrose, trehalose, fructose, xylose, cellobiose and ribose.

15. The method of claim 10, wherein the living cell cultures comprise prokaryotic cells.

16. The method of claim 10, wherein the prokaryotic cells are bacteria or archaea.

17. The method of claim 10, wherein the intracellular levels of the molecule is Monitored in a real time or on-line time frame.

18. The method of claim 10, wherein monitoring the level of molecule comprises calculating a ratio of energy emission values detected from the emission of the donor moiety and the emission from the acceptor moiety.

19. A method of monitoring metabolic flux in a living cell culture comprising expressing a nanosensor in living cells in culture, wherein the nanosensor comprises a donor moiety, an acceptor moiety, and a metabolite binding site, and detecting a change in resonance energy between the acceptor moiety and the donor moiety, thereby monitoring the metabolic flux in the living cell culture.

20. The method of claim 19, wherein the metabolite binding site for the metabolite is on the donor moiety.

21. The method of claim 19, wherein the resonance energy transfer is fluorescence energy transfer, phosphorescence energy transfer, chemiluminescence energy transfer, or bioluminescence energy transfer.

22. The method of claim 19, wherein the metabolite is selected from the group consisting of sugars, amino acids, peptides, organic acids, metals or ions, oxides, hydroxides or conjugates thereof, inorganic ions, amines, polyamines and vitamins.

23. The method of claim 22, wherein the sugars are selected from the group consisting of arabinose, maltose, glucose, galactose, sucrose, trehalose, fructose, xylose, cellobiose and ribose.

24. The method of claim 19, wherein the living cell cultures comprise prokaryotic cells.

25. The method of claim 24, wherein the prokaryotic cells are bacteria or archaea.

26. The method of claim 19, wherein the metabolite is monitored in a real time or on-line time frame.

27. The method of claim 19, wherein the method monitors pentose metabolic flux in living cells.

28. The method of claim 27, wherein the nanosensor is an arabinose sensor.

29. The method of claim 19, wherein the method monitors hexose metabolic flux in living cells.

30. The method of claim 29, wherein the nanosensor is a glucose sensor.

31. The method of claim 19, wherein the cells are genetically engineered to produce biofuel.

32. The method of claim 31, wherein the cells are prokaryotes or a eukaryotes.

33. The method of claim 31, wherein the cells are selected from the group consisting of bacteria, archaea, or eukarya.

34. The method of claim 19, wherein monitoring the metabolic flux comprises calculating a ratio of energy emission values detected from the emission of the donor moiety and the emission from the acceptor moiety.

35. A device for measurement of resonance energy transfer in a living cell, wherein the device comprises a resonance energy transfer (RET) detection unit, a reference unit, and a calibration unit.

36. The device of claim 35, wherein the RET detection unit comprises:
a first filter through which the light from the light source passes to excite a donor moiety in the living cell;
a second filter through which light emitted from the donor moiety passes to reach the detector;
a third filter through which light emitted from the acceptor moiety passes to reach the detector;
a first detector for measuring the emitted light intensity of the donor after excitation of the donor; and
a second detector for measuring the emitted light intensity of the acceptor after excitation of the donor.

37. The device of claim 36, wherein the reference unit comprises:
a light source for excitation of molecules;
a third filter through which light emitted from the acceptor moiety passes to reach the detector;
a fourth filter through which light emitted from the light source passes to excite an acceptor moiety in the living cell; and
a third detector for measuring the emitted light intensity of the acceptor after excitation of the acceptor.

38. The device of claim 37, wherein the calibration unit comprises:
an injector with a container with an analyte stock solution; and
a valve that can close off the device.

39. A device for measurement of resonance energy transfer in a living cell, wherein the device comprises
a light source for excitation of molecules;
a filter through which the light from the light source passes to excite a donor moiety in the living cells;
an optical component that disperses the emitted light from the donor and the acceptor; and
a detector for recording the spectrum of the dispersed light.

40. The device of claim 35, wherein the device measures resonance energy transfer in a real time or on-line time frame.

41. The device of claim 35, wherein the detector comprises:
at least two photo multiplier detectors; and
a processor which processes the signals from the at least two photo multiplier detectors.

42. The device of claim 41, wherein the processor is calculates a ratio of light intensity values comprising a ratio of energy emission values detected from the emission of the donor moiety and the emission from the acceptor moiety.

43. The device of claim 35, wherein the light source is selected from the group consisting of a LED, a mercury lamp, a xenon lamp, and a laser.

44. The device of claim 43, wherein the light source is a LED.

45. The device of claim 41, wherein the photomultiplier detector is a light sensitive diode or a CCD chip.

46. The device of claim 35, wherein the device further comprises an injector for calibration with external ligands.

47. A method of monitoring the concentration of a sugar during fermentation or hydrolysis processes comprising mounting the device of claim 35 in a fermenter and using the device to monitor the concentration of the sugar.

48. A method of monitoring the concentration of a sugar during fermentation or hydrolysis processes comprising passing a fermenter culture through the device of claim 35 and using the device to monitor the concentration of the sugar.
